(19) United States
(12) Patent Application Publication
(10) Pub. No.: US 2006/0127310 A1
(43) Pub. Date: Jun. 15, 2006

(54) Amplification of biotin-mediated targeting

(75) Inventors: Gregory Russell-Jones, NSW (AU); John Mcewan, NSW (AU)

Correspondence Address: FOLEY AND LARDNER LLP;SUITE 500, 3000 K STREET NW, WASHINGTON, DC, 20007, US

(73) Assignee: Access Pharmaceuticals Australia PTY LTD.

(21) Appl. No.: 10/535,269

(22) PCT Filed: Nov. 21, 2003
(86) PCT No.: PCT/AU03/01557

§ 371 Date: Nov. 21, 2005

(30) Foreign Application Priority Data
Nov. 21, 2002 (AU) 2002953073

(51) Int. Cl. A61K 51/00; A61K 39/395; C07K 16/46

(52) U.S. Cl. 424/1.49; 424/9.6; 424/178.1; 530/391.1; 977/927

(57) ABSTRACT

The present invention relates to the delivery of drug, peptide and protein pharmaceuticals using a biotin-mediated uptake system. More particularly the invention relates to the amplification of active substance delivery with the biotin uptake system using a biotin-active substance-polymer conjugate or a biotin-nanoparticle conjugate. The invention also relates to processes for preparing the conjugates, pharmaceutical and diagnostic compositions containing same and methods of diagnosis and treatment involving the conjugates.

### FIELD OF THE INVENTION

The present invention relates to the delivery of drug, peptide and protein pharmaceuticals using a biotin-mediated uptake system. More particularly the invention relates to the amplification of active substance delivery with the biotin uptake system using a biotin-active substance-polymer conjugate or a biotin-nanoparticle conjugate. The invention also relates to processes for preparing the conjugates, pharmaceutical and diagnostic compositions containing same and methods of diagnosis and treatment involving the conjugates.

### BACKGROUND OF THE INVENTION

In conventional cancer chemotherapy, to obtain a linear increase in cancer-cells kill rates it is often necessary to exponentially increase the dosage of cytotoxic drugs. This in turn leads to an undesirable increase in non-specific cytotoxicity of bystander, healthy cells. In order to reduce the effect of the high dose of toxin on normal, healthy tissues, it is often necessary to repeatedly deliver a smaller dose of cytotoxin, which often leads to the survival of a small fraction of drug-resistant cells.

Attempts have been made to increase the dose of cytotoxic agent delivered to the tumor cell, through the use of specific targeting agents such as monoclonal antibodies specific for “tumor-antigens”. In many cases it has been found that the resultant antibody-drug conjugate is highly immunogenic, and thus may lead to an antibody response against the conjugate, which means that treatment must be halted. For this reason small, poorly immunogenic molecules, which have a high specificity for tumour cells, have been sought as alternatives to antibody-mediated targeting agents.

Several candidate targeting agents have recently been identified, and these agents include vitamins, which are essential for the growth of rapidly dividing cells such as tumours. Two such vitamins, vitamin B12 and folic acid, have been shown to target a small subset of aggressive tumour cell lines. Russell-Jones and co-workers have previously described the use of vitamin B12 and folic acid as targeting agents for the delivery of polymers and nanoparticles containing, or linked to, pharmaceuticals both for oral delivery and also for cancer therapy (see for example WO00/66090, WO00/66091 and WO94/27641).

However, the receptivity of cancer cells to these vitamins is variable as a consequence of differential upregulation of the cell-surface receptors for these vitamins. In particular, the aforementioned inventors have found, using in vitro studies involving numerous tumor cell-lines, that many tumor cells upregulate either vitamin B 12 or folate receptors, but there are relatively few examples where receptors for both of these vitamins are simultaneously upregulated. The efficacy of vitamin B12 and folate-targeted chemotherapy is thus not optimal for many types of cancers. It would be desirable, therefore, to be able to utilize a targeting agent which could target a greater proportion of tumor cell-lines, to generate a targeted chemotherapy regime with broader utility in the treatment of cancer.

Biotin is one of the water-soluble, B-group vitamins and is used for fat, protein and carbohydrate metabolism, cell growth and fatty acid production. Biotin has been employed in the laboratory as a trace and in imaging studies with IGG monoclonal antibodies. Biotin conjugates are reported in the literature, and many of the biotin conjugates of the prior art rely on biotin's very high affinity for avidin and streptavidin. Biotin/avidin and biotin/streptavitin systems have been developed for in vitro assay systems as well as in vivo targeting. In the latter case, biotin is bound to a targeting agent, such as an antibody or antibody fragment, which targets a specific area of the body. Circulating biotin-conjugate is allowed to clear, or clearance is accelerated through the use of another material. Finally, the material to be targeted, be it a therapeutic or diagnostic agent, and which is covalently linked to avidin or strepavidin, is administered, and the powerful affinity of avidin or streptavidin for biotin ensures that a high proportion of the injected dose of the avidin/streptavidin conjugate is targeted to, and remains in, the region(s) of the body containing the targeted biotin-conjugate.

Limitations exist in the use of biotin to target to tumor cells when using small-molecule, biotin-conjugate constructs (for example, one molecule of drug for each molecule of biotin). The dose deliverable is small because of the low receptor density, and, because of the small size of the biotin-drug conjugate, they are readily excreted in the kidneys and re-absorbed in the proximal tubules, where there is high density of biotin receptors. This leads to rapid removal of the conjugates from the circulation as well as undesirable accumulation of biotin-drug conjugates in the kidney. These limitations are demonstrated by prior art technology in U.S. Patent Appl No. 20020049154, which teaches the use of biotin and other targeting agents to deliver therapeutic molecules within the body. The pharmaceutical constructs disclosed are limited by size, and furthermore, only one molecule of the active agent is covalently bonded to the biotin.

Cancer and related diseases remain a leading cause of death in today's society. Accordingly there is a strong need to identify new, improved, better and/or alternative pharmaceutical compositions and agents for its treatment, amelioration and prevention. There is a further need for chemotherapeutic agents which address some of the undesirable side effects of known agents. There is also a need for different therapies to be available to physicians to combat the numerous and various types of cancers and to provide new options for treatment to address issues of tolerance of proliferating cells to the existing chemotherapeutic agents and treatment regimes. In addition there is a need for broad-spectrum chemotherapeutics in the field of cancer therapy.

Accordingly it is a preferred object of the present invention to provide pharmaceutical and diagnostic compositions and methods for the diagnosis, treatment, amelioration or prophylaxis of disease by the amplification of active substance delivery to biological targets. The present invention also seeks to provide diagnostic and pharmaceutical compositions and methods for targeting neoplastic cells for treatment, which compositions and methods provide improved cell activity in terms of targeting function and/or improved delivery of toxic and/or diganostic agents.

### SUMMARY OF THE INVENTION

Surprisingly, it has been found by the present inventors that biotin conjugates are able to act as targeting agents for the delivery of macromolecules to many biological targets associated with disease, including cancerous cells and tumours, sites of inflammation, and macrophages and dendritic cells. The biotin conjugates of the invention are large molecular weight complexes incorporating biotin or analogues thereof and an active agent to be delivered. The biotin conjugates of the invention most preferably involve polymer or nanoparticle technology suitable for the amplified delivery of the active agent

The invention further relates to the surprising observation that the vitamin, biotin, is able to target a much wider range of tumours than either vitamin B12 or folate. It is unexpected that biotin-drug conjugates would have such marked activity and wide application to biological targets including cancerous cells and tumours, sites of inflammation, and macrophages and dendritic-cells. This is because uptake of biotin is thought to occur through the sodium dependent multi-vitamin transporter (SMVT), and consequently, while small molecules may be co-transported, large structures such as polymer-drug conjugates cannot be transported.

Simple conjugates of one targeting molecule with one molecule of an active agent have significant drawbacks, for reasons discussed above. The above-mentioned limitations are addressed by incorporating many molecules of the active agent (eg drug) within the conjugates of the invention, such that the biotin targeting effect is amplified by the provision of many more molecules of the active agent per biotin-receptor interaction.

Conjugate-mediated amplification of the targeted drug delivery can be achieved either by attaching both the active agent and biotin (or biotin analog) to a high molecular weight polymer, or incorporation of the active agent within or on the surface of a nanoparticle, the nanoparticle being coated with biotin or an analogue thereof. Thus, amplification of active agent delivery can occur by a macromolecular conjugates such as a polymer or nanoparticle to which biotin (or an analog) is attached in such a way that it is able to bind to biotin receptors expressed on cell surfaces. Accumulation of the macromolecular biotin-active agent conjugate in the kidneys is also minimised due to the large size.

The biotin conjugates of the invention are particularly suitable for parenteral delivery to tumors as they can utilize the biotin receptor system for binding and uptake, and have the aforementioned advantage of amplifying the amount of active agent which can be delivered via the biotin uptake mechanism, as well as minimising or avoiding targeting to the kidneys by virtue of their size. According to one aspect of the invention there is provided a conjugate comprising at least one biotin targeting molecule or an analog thereof, in association with an active substance and a support for the amplified delivery of the active substance.

The conjugates of the invention preferably involve the use of polymers or nanoparticles as the support for the active substance and biotin-targeting agent. Preferably, the nanoparticle is a nanosphere or a nanocapsule.

The conjugates of the invention comprise at least one targeting molecule (TM) which is a biotin molecule, or analogue thereof, wherein the ability of the targeting molecule to undergo the binding reactions necessary for uptake and transport of biotin in a vertebrate host and the activity of the active substance are substantially maintained, following incorporation and/or following biological release of the active substance from the polymer, nanoparticle, or nanosphere.

Preferably, the biotin or biotin analogue is electrostatically or covalently-linked to the polymer, or coats the surface of the nanoparticle. The active agents of the nanoparticle may be enclosed by the nanoparticle or may coat the surface of the nanoparticle.

In a preferred embodiment of the invention the biotin-targeting moiety is in itself pharmaceutically active, such as by being cytotoxic or having anti-inflammatory activity.

The polymeric conjugates of the invention have the general formula:
(B−Q)n−P−(Q′−A)m

wherein B is biotin, or an analogue thereof, which is a targeting molecule which will bind to a surface biotin receptors on a cell, and where
n, the molar substitution ratio of B in the conjugate, is in the range from 1.0 to 50.0;
P is a pharmaceutically acceptable linear, branched, or dendritic polymer;
A is a diagnostic or pharmaceutically active substance;
m, the molar substitution ratio of A in the conjugate, is in the range from 1.0 to 1000; and
Q and Q′ are independently a covalent bond, or a spacer compound linking biotin, P and A by covalent bonds.

In a further aspect, there is provided a process for synthesising the polymeric conjugates of the invention, comprising one or more of the following steps:
a) reacting the active substance with the polymer to form said conjugate:
b) chemically modifying the active substance to provide at least one functional group capable of forming a chemical linkage, and reacting the active substance and polymer to form said conjugate:
c) chemically modifying the TM to provide at least one functional group capable of forming a chemical linkage and reacting the carrier and polymer to form said conjugate:
d) chemically modifying the active substance and the polymer to provide functional groups capable of forming a chemical linkage, and reacting the active substance and polymer to form said conjugate:
e) reacting the active substance with at least one cross-linking agent and reacting the active substance of polymer to form said conjugate:
f) reacting the TM with at least one cross-linking agent and reacting the polymer and TM to form said conjugate:
g) reacting the active substance and polymer with at least one cross-linking agent and reacting the active substance and polymer to form said conjugate:
h) reacting the active substance directly with a polymeric support to form an intermediate containing one or more molecules of the active substance linked to the polymer, and subsequently coupling the polymer-active substance intermediate to one or more targeting molecules:
i) coupling one or more TM molecules to a polymeric support and subsequently reacting the carrier-polymer intermediate with one or more molecules of the active substance to give a final conjugate containing one or more molecules of the active substance.

In another aspect of the invention there is provided a process for the production of a polymeric conjugate having the general formula:
(B−Q)n−P−(Q′−A)m

wherein B, Q, P, Q′, A, n and m are as defined above, said process selected from:
a) reacting A with P to form an intermediate complex, and thereafter reacting the intermediate conjugate with biotin;
b) reacting B with P to form an intermediate complex and thereafter reacting the intermediate conjugate with A;
c) the process of step a) or step b) wherein one or more of B, P or A are modified to provide at least one functional group capable of forming a chemical linkage prior to coupling with the other reactants; or
d) reacting one or two of B, P or A with Q and/or Q′ prior to coupling with the other reactants.

In a further aspect of the invention there is provided a method for the modification of a polymeric support to introduce functional groups capable of reacting either directly with the active substance or with a chemically-modified form of the active substance. The resulting polymer-active substance intermediate contains one or more molecules of the active substance, said intermediate being suitable for coupling to the TM to give a conjugate capable of amplified delivery of the active substance.

The invention also provides a process for the production of the nanoparticle conjugates of the invention, comprising one or more of the following steps:
(a) reacting nanospheres with biotin or a biotin analogue to form the conjugate;
(b) chemically modifying the biotin molecule or analogue thereof to provide at least one functional group capable of forming a chemical linkage, and reacting nanospheres and the modified TM to form the conjugate;
(c) reacting nanospheres with at least one cross-linking agent to prepare “activated” nanoparticles which are reacted with a TM to form the conjugate;
(d) reacting a TM with at least one cross-linking agent and reacting the nanospheres with the reacted TM to form the conjugate;
(e) reacting nanospheres and a TM with at least one cross-linking agent to form the conjugate;
(f) reacting nanospheres with at least one cross-linking agent, reacting a TM with at least one cross-linking agent and reacting the reacted nanospheres and the reacted TM to form the conjugate; or
(g) reacting a TM with at least one cross-linking agent to prepare an analogue which is reacted with a hydrophobic moiety to form a hydrophobic derivative of the TM; and then incubating the hydrophobic derivative of the TM with the nanosphere in such a manner that the nanosphere is coated hydrophobically with the TM.

According to another aspect of the invention there is provided a diagnostic or pharmaceutical composition which comprises a conjugate of the invention in association with a pharmaceutically acceptable carrier or diluent.

According to another aspect of the invention there is provided a method for the treatment, prophylaxis or amelioration of disease, which comprises the step of administering to a subject a therapeutically effective amount of a conjugate or composition of the invention.

In a preferred embodiment the disease is a form of cancer.

In a further preferred form, the disease is an inflammatory disease.

The conjugates of the invention can be used to stimulate macrophages and dendritic cells with antigens as the active agent through targeting of these complexes of biotin and antigen to biotin receptor positive cells. Moreover, the conjugates of the invention can be used to target macrophages with cytotoxic agents to reduce the severity of macrophage-mediated events in diseases such as psoriasis, colitis, Crohn's disease, multiple sclerosis, graft-versus-host reaction and rheumatoid arthritis.

Thus, according to another aspect of the invention there is provided a method for stimulating macrophages or dendritic cells with an antigen by contacting the macrophage or dendritic cell with a conjugate of the invention, wherein the active agent is an antigen and the macrophage or cells to be contacted are biotin receptor positive.

In a further embodiment of the invention the conjugates can be used to deliver anti-parasitic drugs to macrophages. Such processes can be used in the treatment of intracellular parasites such as malaria, salmonella, and leishmania.

In another embodiment of the invention, the conjugates can be used to enhance the transfer of the drug from the intestinal lumen to the bloodstream.

In a further embodiment, the invention provides a conjugate suitable for imaging of tumours or inflammatory conditions, the conjugate comprising more than one imaging agent linked to a polymer, or more than one imaging agent which is incorporated within and/or coated on the surface of a nanosphere or nanoparticle, wherein the polymer, nanosphere or nanoparticle is linked to at least one targeting molecule which is a biotin molecule, or analogue thereof, wherein the ability of the targeting molecule to undergo the binding reactions necessary for uptake and transport of biotin in a vertebrate host and the activity of the imaging are substantially maintained, following incorporation and/or following biological release of the active substance from the polymer, nanoparticle, or nanosphere.

Furthermore, the invention also provides a conjugate having a biotin molecule, or analogue thereof, as a first targeting molecule, and one or more second targeting molecules, wherein the ability of the first and second targeting molecules, individually or combined, provide the binding reactions necessary for uptake and/or transport of biotin in a cell and/or provide for release and/or promote a biological activity of the active substance in a cell.

According to another aspect of the invention there is provided the use of a conjugate of the invention in the manufacture of a medicament for the diagnosis and/or treatment of disease.

According to another aspect of the invention there is provided the use of biotin or an analogue thereof in the manufacture of a conjugate of the invention.

According to another aspect of the invention there is provided an agent for the diagnosis, treatment, prophylaxis or amelioration of a disease which agent comprises a conjugate of the invention.

These and other aspects of the invention will become evident from the description and claims which follow, together with the accompanying drawings.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

### BRIEF DESCRIPTION OF THE DRAWINGS

FIG. 1 shows cryostat sections of P815 tumor cells taken from mice, 6 hours post Rhodamine-HPMA injection, showing accumulation of this polymer using fluorescent microscopy.

FIG. 2 shows an increased uptake of FITC fluorescent labelled polymers in ascites cells from L1210FR tumors using biotin as a target molecule.

FIG. 3 shows an increased uptake of FITC and TRITC fluorescent labelled polymers in ascites cells from L1210FR tumors using biotin as a target molecule.

FIG. 4 shows an increased uptake of Rhodamine-HPMA polymer using biotin as target molecule in Ov2008 tumor cells.

FIG. 5 shows an increased uptake of Rhodamine-HPMA polymer using biotin as target molecule in RENCA tumor cells.

FIG. 6 shows an increased uptake of Rhodamine-HPMA polymer using biotin as target molecule in 4T1 tumor cells.

FIG. 7 shows an increased uptake of Rhodamine-HPMA polymer using biotin as target molecule in JC tumor cells.

FIG. 8 shows an increased uptake of Rhodamine-HPMA polymer using biotin as target molecule in MMTO60562 tumor cells.

FIG. 9 shows a growth of Colo-26 tumours following treatment with polymer-linked doxorubicin (Dox).

FIGS. 10a and 10b show a plot of tumour growth following treatment with Dox-TP-HPMA-Colo-26. The data depicts mean.

### DETAILED DESCRIPTION OF THE INVENTION

The conjugates of the present invention relate to a support to which an active agent and a biotin molecule, or analogue thereof, are associated or conjugated. These biotin complexes are directed to biological targets having an affinity for biotin, and are particularly suitable for parenteral delivery to tumours, cancerous cells, sites of inflammation, and to macrophages and dendritic cells. The conjugates of the invention have the advantage of increasing the amount of active agent which can be delivered via a biotin uptake mechanism, as well as minimising or avoiding targeting to the kidneys by virtue of their size. The support is preferably a polymer, nanoparticle, or nanosphere. Below are separate descriptions for polymers and for nanoparticles/nanospheres:

Polymers

The polymer conjugates of the present invention are targeted to cancer cells using biotin or analogues thereof as the targeting molecule. Once the drug-polymer conjugate has reached its target tissue, the conjugate binds to a cell-surface receptor and initiates receptor-mediated endocytosis, which transports the conjugate to the cell interior. The pendant drug may be released by the action of lysosomal enzymes, by cleavage of a disulfide linked drug by intracellular glutathione or otherwise. These polymeric conjugates may be used for oral delivery of the drug to the circulatory or lymphatic drainage system. Preferably, the polymeric conjugates and compositions of the invention relate to targeting the drugs/pharmaceuticals or imaging agents to sites of disease, especially tumor/cancer cells.

In a further embodiment the polymer conjugates of the present invention have been targeted to macrophages using biotin or analogues thereof as the targeting molecule. Once the drug has reached an inflammatory site, the conjugate is endocytosed by the target macrophage and the pendant drug may be released by the action of lysosomal enzymes, by cleavage of a disulfide linked drug by intracellular glutathione, or by the acid environment within intracellular compartments such as endosomes and lysosomes, or other means.

While it is the belief of the inventors that the therapeutic benefit provided by the polymer conjugates of this invention is provided by the above stated mechanisms, it is possible that other mechanisms of action may provide benefit, and this invention is not restricted to any one mechanism of action.

The polymer, P (as defined above), of the present invention can be any pharmaceutically acceptable polymer. The polymer is able to attach to at least one TM and to at least one, but preferably a multiplicity, of active substance molecules. The polymer may be naturally occurring or synthetic or a mixture thereof, and can be linear, branched, or dendritic.

Suitable polymers for substitution with biotin and modification according to the invention, include, but are not limited to, poly[N-(2-hydroxypropyl)-methacrylamide], dextran, chondroitan sulfate, water soluble polyurethanes formed by covalent linkage of PEG with lysine, poly(glutamic acid), poly(hydroxypropyl glutamine) and branched chain polypeptides formed by the dual modification of the γ- and α-amino groups of lysine during the peptide synthesis, as well as dendrimers and PEG-dendrimers, dextran, dextrin, glycosaminoglycans, carboxymethylcellulose, polylactic acid, polyglutamic acid, poly[lactide-co-glycolide], polyhydroxyethymethacrylate (poly-HEMA), human serum albumen (HSA), and other such biodegradable, or non-biodegradable polymers. Such polymers may have multiple amino-termini, to which can be conjugated a plurality of the pharmaceutical or drug to be delivered. The polymers can also be formed with multiple cystines, to provide free thiols, or multiple glutamates or aspartates, to provide free carboxyls for conjugation using suitable carbodiimides. Similarly the polymer can contain multiple histidines or tyrosines for conjugation. The polymer may have multiple hydroxyl groups suitable for modification, or alternatively may contain vicinal hydroxyl groups suitable for oxidation with reagents such as periodic acid, such that chemistry well known in the art can be used to conjugate the TM and drug. The polymer may also have multiple carboxy groups for conjugation using suitable carbodiimides.

Preferably the linkage to the polymer, or the polymer to which the pharmaceutical is linked, should be degradable or biodegradable. Potentially biodegradable polymers include dextran and its derivatives, as well as dextrin, amino acid polymers such as polylysine, poly-glutamic acid, alginate, heparin sulphate, and other sulphated polysaccharides, gelatin, glycosaminoglycans, poly[lactide-co-glycolide], polyhydroxyethymethacrylate (poly-HEMA), HSA or other similar proteins.

Non-biodegradable polymers may also be employed in the present invention and include poly[N-(2-hydroxypropyl)methacrylamide], to which is attached biodegradable side chains such as those containing ester linkages, or amino acid sequences cleavable within lysosomal vacuoles ie. Gly-Phe-Leu-Gly (Rihova, B. and J. Kopecek 1985 Biological properties of targetable poly[N-(2-hydroxypropyl)-methacrylamide]-antibody conjugates. J. Control Rel., 2:289-310]. Other amino acid spacers cleavable by intracellular proteases include Gly-Phe-Ala; Gly-Phe-Ala-Gly; Gly-Phe-Tyr-Ala; and Gly-Phe-Tyr-Ala-Ala, Ala-Leu-Ala-Leu [Rejmanova, P., Obereigner, B., and Kopecek, J. 1981 Makromol. Chem. 182:1899-1915].

The preferred TM is biotin, or an analogue of biotin, either of which may be adapted provided that binding to cell surface biotin receptors at disease sites is still possible. Biotin is most easily covalently attached to a ligand, or the polymer, via its carboxylic acid moiety. Alternatively, the TM can be modified to have charged groups of opposite charge to functional groups on the polymer such that the TM is bound by non-covalent (electrostatic, H-bonded, and hydrophobic bonding) forces.

Suitable analogues of biotin, according to the invention include, but are not limited to biotin, iminobiotin, Biocytin hydrazide, Biotin hydrazide, biocytin, 5-(Biotinamido)pentylamine, Sulfo-NHS(n-Hydroxysuccinimidyl)-Biotin, Sulfo-HNS-hexanyl-biotin (Sulfo-NHS-LD-Biotin), NHS-Biotin, Pentafluorophenyl-biotin, Pentafluorophenyl-polyethylenoxide-biotin, NHS-biotin Trifluoroacetamide, NHS-Iminobiotin trifluoroacetamide, Maleimido-polyethylenoxide biotin, Maleimido-polyethylenoxide iminobiotin, desthiobiotin, chloracetyl-biotin.

Further biotin analogues include 3-(N-Maleimido-propionyl)biocytin:a thiol-specific biotinylating reagent, alpha-dehydrobiotin, Z- and E-4,5-dehydrodethiobiotin, norbiotinamine, d1-4 xi-(4-carboxybutyl)-5-carbethoxy-cis-hexahydropyrrolo (3,4-d)imidazol-2-one (N-carbethoxyazabiotin), d1-4xi-(2-carboxyethyl)-cis-hexahydropyrrolo-[3,4-d]imidazol-2-one (bisnorazabiotin), bis-allyloxycarbonyl biotin aldehyde, carboxybiotin, methyl biotin.

In one embodiment of the invention the linkage joining the pharmaceutical, or the biotin to the polymer is a disulfide bond. In a further embodiment of the invention the linkage joining the pharmaceutical, or the biotin to the polymer is an ester linkage. In yet another embodiment of the invention the linkage joining the pharmaceutical or the biotin to the polymer is a γ-glutamyl-ε-lysine bond. In yet another embodiment of the invention the linkage joining the pharmaceutical or the biotin to the polymer is a diazo-linkage. In yet a further example the bond linking the drug to the polymer is an acid labile linker, such as that formed with aconitic acid or via a hydrazone linkage.

The spacer groups Q and Q′ are optional. When they are absent the biotin TM, and/or the active substance A are linked to polymer P by a direct covalent or electrostatic bond. Spacer groups are introduced either to improve the biotin receptor affinity of the biotin conjugate or to overcome problems in the coupling of the carrier, biotin, and/or the active substance A arising from unfavourable steric interactions between the biotin and A with the polymer P, or to increase the bioactivity of A in the conjugate. The spacer groups may also act as linking agents, being bi-functional compounds with selected functional groups on each end to react with suitable functional groups located on the polymer, and also on the biotin carrier molecule and/or on the pharmaceutically active substances.

Suitable extended spacers for the conjugation of the pharmaceutical or biotin to the polymer matrix include: disuccinimidyl suberate (DSS), bis(sulfosuccinimidyl) suberate (BSS), ethylene glycolbis(succinimidylsuccinate) (EGS), ethylene glycolbis(sulfosuccinimidylsuccinate) (Sulfo-EGS), p-amino-phenylacetic acid, dithiobis(succinimidylpropionate) (DSP), 3,3′-dithiobis(sulfosuccinimidylpropionate) (DTSSP), disuccinimidyl tartarate (DST), disulfosuccinimidyl tartarate (Sulfo-DST), bis[2-(succinimidyloxycarbonyloxy)-ethylene]sulfone (BSOCOES), bis[2-(sulfosuccinimidooxycarbonyloxy)-ethylene]sulfone (Sulfo-BSOCOES), dimethyl adipimidate.2 HCl (DMA), dimethyl pimelimidate.2 HCl (DMP), dimethyl suberimidate.2 HCl (DMS), N-succinimidyl(4-iodoacetyl)aminobenzoate (SIAB), succinimidyl 4-(p-maleimidophyl)butyrate (SMPB).

The active substance to be delivered is preferably a hormone, drug, prodrug, toxin, pharmaceutically active protein, immunogen, or DNA or RNA analogue.

Suitable toxins, according to the invention, include, but are not limited to, ricin, abrin, diphtheria toxin, modecin, tetanus toxin, mycotoxins, mellitin, α-amanitin, pokeweed antiviral protein, ribosome inhibiting proteins, especially those of wheat, barley, corn, rye, gelonin, maytansinoid.

Suitable cytotoxic agents, according to the invention, include, but are not limited to alkylating agents such as chlorambucil, cyclophosphamide, melphalan, cyclopropane; anthracycline antitumor antibiotics such as doxorubicin, daunomycin, adriamycin, mitomycin C, [2-(hydroxymethyl)anthraquinone]; antimetabolites such as methotrexate, dichloromethatrexate: cisplatin, carboplatin, and metallopeptides containing platinum, copper, vanadium, iron, cobalt, gold, cadmium, gallium, iron zinc and nickel. Other agents include DON, thymidine, pentamethylmelamin, dianhydrogalactitol, 5-Methyl-THF, anguidine, maytansine, neocarzinostatin, chlorozotocin, AZQ, 2′deoxycoformycin, PALA, AD-32, m-AMSA and misonidazole, deferoxamine, ferrioxamine, iron-basic porphine.

Additional cytotoxins which may be employed in the conjugates of the invention include epirubicin, platinum derivatives, including cis-Platin, CarboPlatin, oxaliplatin, multinuclear platinate species including BBR3464 and BBR3005, transdiamminedichloroplatinum (II) (Transplatin), chlorodiethylenetriammineplatinum (II), Platinum IV compounds, spiroplatin, platin-phosphine derivatives, calicheamycin, dolastatin derivatives, including auristatin, monomethylauristatin.

Suitable imaging agents, according to the invention include, but are not limited to those described by Molecular Probes (Handbook of fluorescent probes and research products) included by way of reference), such as Rhodamine, fluorescein, Texas red, Acridine Orange, Alexa Fluor (various), Allophycocyanin, 7-aminoactinomycin D, BOBO-1, BODIPY (various), Calcien, Calcium Crimson, Calcium green, Calcium Orange, 6-carboxyrhodamine 6G, Cascade blue, Cascade yellow, DAPI, DiA, DiD, Dil, DiO, DiR, ELF 97, Eosin, ER Tracker Blue-White, EthD-1, Ethidium bromide, Fluo-3, Fluo4, FM1-43, FM4-64, Fura-2, Fura Red, Hoechst 33258, Hoechst 33342, 7-hydroxy-4-methylcoumarin, Indo-1, JC-1, JC-9, JOE dye, Lissamine rhodamine B, Lucifer Yellow CH, LysoSensor Blue DND-167, LysoSensor Green, LysoSensor Yellow/Blu, Lysotracker Green FM, Magnesium Green, Marina Blue, Mitotracker Green FM, Mitotracker Orange CMTMRos, MitoTracker Red CMXRos, Monobromobimane, NBD amines, NeruoTrace 500/525 green, Nile red, Oregon Green, Pacific Blue. POP-1, Propidium iodide, Rhodamine 110, Rhodamine Red, R-Phycoerythrin, Resorfin, RH414, Rhod-2, Rhodamine Green, Rhodamine 123, ROX dye, Sodium Green, SYTO blue (various), SYTO green (Various), SYTO orange (various), SYTOX blue, SYTOX green, SYTOX orange, Tetramethylrhodamine B, TOT-1, TOT-3, X-rhod-1, YOYO-1, YOYO-3.

Additionally radionuclides can be used according to the invention either as imaging agents or as pharmaceutically active substances. These radionuclides include, but are not limited to radioactive species of Fe(III), Fe(II), Cu(II), Mg(II), Ca(II), and Zn(I1) Indium, Gallium, Technetium, such as ^{99m}Technetium. ¹¹¹Indium, ¹⁸⁶Re, ¹⁸⁶Re ^{66,67,68}Ga, ⁹⁰Y ¹⁴⁹Pm, ¹⁷⁷Lu, ²⁷Mg, ⁴⁷Ca, ⁶⁴Cu. Also are included metal ions generally used for chelation in paramagnetic T1-type MIR contrast agents, and include di- and tri-valent cations selected from the group consisting of copper, chromium, iron, gadolinium, manganese, erbium, europium, dysprosium and holmium. Metal ions that can be chelated and used for radionuclide imaging according to the invention, include, but are not limited to metals selected from the group consisting of gallium, germanium, cobalt, calcium, indium, iridium, rubidium, yttrium, ruthenium, yttrium, technetium, rheniumn, platinum, thallium and samarium. Additionally metal ions known to be useful in neutron-capture radiation therapy include boron and other metals with large nuclear cross-sections. Also included are metal ions useful in ultrasound contrast, and X-ray contrast compositions.

Suitable metal chelators according to the invention include, but are not limited to HYNIC (2-hydrazinonicotinic acid), HYBIN, DTPA (N-diethylenetriaminopentaacetic acid), cyclams, DOTA and its derivatives (1,4,7,10-tetraazacyclododecane- N,N′,N″, N″′-tetraacetic acid), TETA. TETA (1,4,8,11-tetraazacyclotetradecane-1,4,8,-11-tetraacetic acid), NOTA. NOTA (1,4,7-triazacyclononane-1,4,7-triacetic acid),

Suitable cross-linking agents for use in the preparation of thiol-cleavable biodegradable linkers include N-succinimidyl 3-(2-pyridyldithio)propionate (SPDP), iminothiolane, sulfosuccinimidyl 6-[3-(2-pyridyldithio) propionamido] hexanoate (Sulfo-LC-SPDP), succinimidyl 6-[3-(2-pyridyldithio) propionamido] hexanoate (LC-SPDP), sulfosuccinimidyl 6-[α-methyl-α-(2-pyridyldithio) toluamido]hexanoate (Sulfo-LC-SMPT), 1,4-di[3′-(2′-pyridyldithio)propionamido]butane (DPDPB), 4-succinimidyloxycarbonyl-α-methyl-α-(2-pyridyldithio)-toluene (SMPT), dimethyl 3,3′dithiobispropionimidate.2 HCl (DTBP).

Additional linkers include those consisting of or containing 5-benzoyl-valeric acid, valine-citrilline dipeptide, phenylalanine-lysine dipeptide, Gly-Phe-Leu-Gly.

It is within the scope of this invention to deliver other active substances or utilize other linkers known in the art.

Furthermore, it is within the scope of this invention to deliver two or more different active substances by attaching said two or more active compounds to the polymer by the methods described above.

Furthermore, it is within the scope of this invention to utilize one or more TM in addition to biotin (or a biotin analog) by attaching to the polymer the two (or more) different TMs. Additional TMs include, but are not limited to, vitamin B12 and folic acid (and folic acid derivatives).

Nanaoparticles and Nanospheres

Two basic forms of nanoparticles have been developed, nanocapsules (or microcapsules) and nanospheres (or nanospheres), for enclosing, holding or containing an active substance. The terms “nanoparticle”, “nanocapsule”, and “nanosphere” as used throughout the specification refer to a material or construct ranging in size from 1 nanometer to 100 micrometers in size, which may be spherical or have some other shape.

The nanoparticle conjugates of the present invention have been targeted to cancer cells using biotin or analogues thereof as the targeting moiety. The drug may be released from the nanoparticle to the circulatory or lymphatic drainage system, and most preferably to the target tissue of the host. Whilst it is possible that these nanoparticle conjugates could be used for oral delivery of the drug to the circulatory or lymphatic drainage system in general, the products of this invention preferably relate to targeting the drugs, pharmaceuticals to the sites of disease, especially tumor/cancer cells.

The active substance to be delivered is preferably a hormone, drug, prodrug, toxin, pharmaceutically active protein, immunogen, or DNA or RNA analogue.

In essence the nanoparticles can be formed by any number of methods, several of which are outlined below:

(i) Solvent Evaporation

In which a compound which is soluble in one solvent is dispersed into another miscible solvent and the first solvent is evaporated off. Particles formed in this fashion have been used to administer (parenterally) a number of water insoluble compounds. An example of such a system would be the formation of polyalkylcyanoacrylate nanocapsules in which the anticancer agent, 5-fluorouracil is entrapped.

(ii) Desolvation

In this method a compound is contained in a liquid in which it is soluble (the solvent) and a second liquid (which is miscible with the first liquid, but in which the compound is not soluble) is added to the solvent. As more of the second liquid is added the compound becomes desolvated. During the process of desolvation the compound rich phase (the coacervate) contains an enriched amount of compound which is dispersed as microdroplets in the compound deficient phase. At this stage the coalesced material can be chemically crosslinked by a suitable crosslinking agent to form micro- or nano-particles. Nanoparticles of gelatin or BSA can be prepared in this way. Solutions of these proteins are dessolvated by the addition of sodium sulfate, or ammonium sulfate solutions. At the point of desolvation there is an increase in turbidity, at which time the nanoparticles can be formed by the addition of a suitable cross-linker such as glutaraldehyde or butanedione. Alternatively a biodegradable cross-linker could be employed, such as a linker containing a disulfide bond, an azo-bond, or an esterase cleavable bond.

(iii) Complex Coacervation

In this procedure two polyelectrolytes having opposite charge are mixed in aqueous medium so that a spontaneous liquid/liquid phase separation occurs. The phenomenon is limited to polymers having a suitable ionic charge density and chain length. Typically these nanospheres are formed by the addition of a polyanion such as Gum Arabic, Alginate, or Polyphosphate, to a polycation such as Gelatin. Suitable particles are readily formed by the complexation of gelatin and carboxymethyl cellulose. The rate of release of pharmaceutical from such complexes can be controlled by the addition of a suitable cross-linker such as glutaraldehyde or butanedione. Alternatively a biodegradable cross-linker could be employed, such as a linker containing a disulfide bond, an azo-bond, or an esterase cleavable bond.

(iv) Polymer/Polymer Incompatability

This procedure is based upon the observation that two chemically different polymers dissolved in a common solvent are usually incompatible. Thus the mixture will tend to form two phases. The insoluble phase can be used to coat core particles to form microcapsules. An example would be the precipitation of ethyl cellulose from cyclohexane by the addition of polyethylene.

(v) Interfacial Polymerization

In this technique, two reactants, each dissolved in a mutually immiscible liquid, diffuse to the interface between the two liquids where they react to form a capsule wall. An example of such capsule formation would occur if a mixture of Sebacoyl chloride dissolved in an oil phase and emulsified into an aqueous phase containing ethylenediamine.

Other methods of formation of nanoparticles, nanocapsules, and nanospheres are known in the art, and can be applied for the purpose of constructing nanoparticles for the present invention

In one embodiment, the invention provides a conjugate between biotin and a biodegradable nanosphere in which is trapped a toxin or cytotoxic agent or active substance.

Suitable analogues of biotin, according to the invention include, but are not limited to biotin, iminobiotin, Biocytin hydrazide, Biotin hydrazide, biocytin, 5-(Biotinamido)pentylamine, Sulfo-NHS(n-Hydroxysuccinimidyl)-Biotin, Sulfo-HNS-hexanyl-biotin (Sulfo-NHS-LD-Biotin), NHS-Biotin, Pentafluorophenyl-biotin, Pentafluorophenyl-polyethylenoxide-biotin, NHS-biotin Trifluoroacetamide, NHS-Iminobiotin trifluoroacetamide, Maleimido-polyethylenoxide biotin, Maleimido-polyethylenoxide iminobiotin, Iodoacetyl-biotin, Chloroacetyl-biotin.

Suitable toxins, according to the invention, include, but are not limited to, ricin, abrin, diphtheria toxin, modecin, tetanus toxin, mycotoxins, mellitin, alpha-amanitin, pokeweed antiviral protein, riosome inhibiting proteins, especially those of wheat, barley, corn, rye, gelonin, maytansinoid.

Suitable cytotoxic agents, according to the invention, include, but are not limited to alkylating agents such as chlorambucil, cyclophosphamide, melphalan, cyclopropane; anthracycline antitumor antibiotics such as doxorubicin, daunomycin, adriamycin, mitomycin C, [2-(hydroxymethyl)anthraquinone]; antimetabolites such as methotrexate, dichloromethatrexate: cisplatin, carboplatin, and metallopeptides containing platimun, copper, vanadium, iron, cobalt, gold, cadmium, iron, gallium, zinc and nickel. Other agents include DON, thymidine, pentamethylmelamin, dianhydrogalactitol, 5-Methyl-THF, anguidine, maytansine, neocarzinostatin, chlorozotocin, AZQ, 2′deoxycoformycin, PALA, AD-32, m-AMSA and misonidazole.

Polymers suitable for the formation of nanospheres by solvent evaporation (in liquid drying) include, amongst others, Poly-lactic acid, Poly-(Lactide/co-glycolide), Poly-hydroxybutyrate, Poly-hydroxyvalerate, Poly-(hydroxybutyrate/valerate), Ethyl cellulose, Dextran, Dextrin, Polysaccharides, Polyalkylcyanoacrylate, Poly-methyl-methacrylate, poly(e-caprolactone) and various combinations and co-polymers of the above.

Polymers suitable for the formation of nanospheres by interfacial precipitation/polymerization include, amongst others, EUDRAGITTM; Poly(N′,N″L-lysinediylterephthaloyl); polymers formed by the reaction of Lysine hydrochloride and p-phthaloyl dichloride; by the reaction of acryloylated maltodextrin or acryloylated hydroxyethyl starch with ammonium peroxodisulfate and N,N,N′,N′-tetrarnethylethylenediamine. Nanospheres can also be formed by the polymerization of various diamines such as ethylene diamine, phenylenediamine, toluene diamine, hexamethylene diamine, or diols such as ethylene diol, bisphenol, resorcinol, catechol, pentanediol, hexanediol, dodecanediol, 1,4 butanediol, with diacid chlorides such as sebacoylchloride and adipoyl chloride, or diisocynates such as hexamethylene diisocyanate using the methods fully described in EPA 85870002.4.

Polymers suitable for the formation of nanospheres by polymer phase separation include co-poly(vinyl chloride:vinyl alcohol:vinyl acetate), cellulosic polymers, polyvinyl acetate, polyvinyl alcohol, polyvinylchloride, natural and synthetic rubbers, polyacrylates, polystyrene and the like. Methods to synthesize such nanospheres are fully described in U.S. Pat. No. 4,166,800.

Polymers suitable for the formation of nanospheres by complex coacervation include, amongst others, mixtures of polyanions, such as gum arabic, alginate, carboxymethyl cellulose, carboxymethyl starch, polystyrene sulfonic acid, polyvinyl sulfonic acid, poly-D-glucuronic acid, Poly-pyruvic acid, carrageenan, heparin sulphate, polyphosphate with polycations, such as polylysine, gelatin.

Polymers suitable for the formation of nanospheres by Polymer/Polymer incompatability include, amongst others, ethyl cellulose, Ethylene vinyl acetate polymer, Poly(lactide), or Poly(vinylidene chloride) mixed with polymers such as Polyethylene, Silicone, Polyisobutylene or Polybutadiene.

Other materials suitable for formation of nanospheres include, Starch, Cross-linked Albumen, Polyacrylamide, Cross-linked gelatin and others obvious to those skilled in the art of nanosphere preparation.

The cross-linking agent may contain a disulfide bond or be cleavable by acid, base or periodate. Examples of suitable cross-linking agents include: N-(4-azidophenylthio)phthalimide; 4,4′-dithiobisphenylazide; dithiobis(succinimidylpropionate); dimethyl-3,3′-dithiobispropionimidate.2HCl; 3,3′-dithiobis-(sulfosuccinimidylpropionate); ethyl-4-azidophenyl)-1,3′dithiopropionate; sulfosuccinimidyl-2-(m-azido-o-nitrobenzamido)-ethyl-1,3′-dithiobutyrimuidate.HCl; N-succinimidyl-(4-azidophenyl)-1,3′dithiopropionate; sulfosuccinimidyl-2-(m-azido-o-nitrobenzamido)-ethyl-1,3′-dithiopropionate; sulfosuccinimidyl-2-(p-azidosalicylamido)-ethyl-1,3′-dithiopropionate; N-succinimidyl-3-(2-pyridylthio)propionate; sulfosuccinimidyl-(4-azidophenyldithio)-propionate; 2-iminothiolane; disuccinimidyl tartrate; and bis-[2-(succinimidyloxycarbonyloxy)-ethyl]-sulfone.

Suitable linking of the TM to the nanospheres may be achieved by reaction of the TM with a carbodiimide and N-hydroxysuccinimide (NHS), and then reacting the NHS derivative with a suitable functional group on the nanosphere.

Examples of pharmaceutically acceptable carriers, diluents and excipients for oral delivery include sodium bicarbonate solutions and similar diluents which neutralise stomach acid or have similar buffering capacity, glycols, oils or emulsions; and include medicaments in the form of gels, pastes and viscous colloidal dispersions. The medicament may be presented in capsule, tablet, slow release or elixir form or as a gel or paste. Furthermore the medicament may be presented as a food.

Examples of pharmaceutically acceptable carriers, diluents and excipients for parenteral delivery include saline, glycols, oils or emulsions; and include medicaments in the form of gels, pastes and viscous colloidal dispersions. It is within the scope of this invention to incorporate the active substance within the nanoparticle and/or to coat the active substance on the surface of the particle, provided that the TM bound to the surface of the nanoparticle is available for receptor-binding to cell-surface biotin receptors at the sites of disease.

Furthermore, it is within the scope of this invention to attach the TM to the nanoparticle either by covalent bonding, or by physical coating, in which the TM is bound by a combination of electrostatic, H-bonding and/or hydrophobic bonding.

Furthermore, it is within the scope of this invention to deliver other active substances or utilize other material (from that described above) known in the art for the formation of nanoparticles.

Furthermore, it is within the scope of this invention to deliver two or more different active substances by incorporating and/or coating said two (or more) active compounds within and/or onto the nanoparticle by the methods described above.

Furthermore, it is within the scope of this invention to utilize one or more TM in addition to biotin (or a biotin analog) by attaching to the nanoparticle the two (or more) different TMs. Additional TMs include (but are not limited to) vitamin B12 and folic acid (and folic acid derivatives).

The compositions described herein, when used for the treatment of disease, may conceivably be used with or without the use of other pharmaceutical agents.

Compositions have been described herein possessing a single pharmaceutically-active ingredient, either attached or incorporated. It is within the scope of this invention for compositions to possess a plurality of pharmaceutically-active compounds, their derivatives and/or prodrugs, either attached or incorporated, such combinations of pharmaceutically-active compounds providing an additive or synergistic benefit in the treatment of disease.

The terms “conjugate” and “macromolecular conjugate” are used herein in their broadest sense to include all forms and synthetic stages (ie intermediate conjugates) of the biotin-mediated targeting compounds, compositions, complexes of the invention.

As used herein, the terms “treatment”, “prophylaxis” or “prevention”, “amelioration” and the like are to be considered in their broadest context. In particular, the term “treatment” does not necessarily imply that an animal is treated until total recovery. Accordingly, “treatment” includes amelioration of the symptoms or severity of a particular condition or preventing or otherwise reducing the risk of developing a particular condition.

The amount of the conjugate of the invention which is required in a therapeutic treatment according to the invention will depend upon a number of factors, which include the specific application, the nature of the particular compound used, the condition being treated, the mode of administration and the condition of the patient. The conjugates may be administered in a manner and amount as is conventionally practised. The specific dosage utilised will depend upon the condition being treated, the state of the subject, the route of administration and other well known factors as indicated above. The length of dosing may range from a single dose given once every day or two, to twice or thrice daily doses given over the course of from a week to many months to many years as required, depending on the severity of the condition to be treated or alleviated. It will be further understood that for any particular subject, specific dosage regimens should be adjust over time according to the individual need and the professional judgment of the person administering or supervising the administration of the compositions.

The production of pharmaceutical compositions for the treatment of the therapeutic indications herein described are typically prepared by admixture of the conjugates of the invention with one or more pharmaceutically or veterinary acceptable carriers and/or excipients as are well known in the art.

Examples of pharmaceutically acceptable carriers, diluents and excipients for oral delivery include sodium bicarbonate solutions and similar diluents which neutralise stomach acid or have similar buffering capacity, glycols, oils or emulsions; and include medicaments in the form of gels, pastes and viscous colloidal dispersions. The medicament may be presented in capsule, tablet, slow release or elixir form or as a gel or paste. Furthermore the medicament may be presented as a food. Examples of pharmaceutically acceptable carriers, diluents and excipients for parenteral delivery include saline, glycols, oils or emulsions; and include medicaments in the form of gels, pastes and viscous colloidal dispersions.

In particular, the carrier must, of course, be acceptable in the sense of being compatible with any other ingredients in the formulation and must not be deleterious to the subject. The carrier or excipient may be a solid or a liquid, or both, and is preferably formulated with the compound as a unit-dose, for example, a tablet, which may contain up to 100% by weight of the active compound, preferably from 0.5% to 59% by weight of the active compound. One or more active compounds may be incorporated in the formulations of the invention, which may be prepared by any of the well known techniques of pharmacy consisting essentially of admixing the components, optionally including one or more accessory ingredients. The preferred concentration of active compound in the drug composition will depend on absorption, distribution, inactivation, and excretion rates of the drug as well as other factors known to those of skill in the art.

The formulations of the invention include those suitable for oral, rectal, optical, buccal (for example, sublingual), parenteral (for example, subcutaneous, intramuscular, intradermal, or intravenous) and transdermal administration, although the most suitable route in any given case will depend on the nature and severity of the condition being treated and on the nature of the particular active compound which is being used.

Formulation suitable for oral administration may be presented in discrete units, such as capsules, sachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture such as to form a unit dosage. For example, a tablet may be prepared by compressing or moulding a powder or granules containing the active compound, optionally with one or more accessory ingredients. Compressed tablets may be prepared by compressing, in a suitable machine, the compound of the free-flowing, such as a powder or granules optionally mixed with a binder, lubricant, inert diluent, and/or surface active/dispersing agent(s). Moulded tablets may be made by moulding, in a suitable machine, the powdered compound moistened with an inert liquid binder.

Formulations suitable for buccal (sublingual) administration include lozenges comprising the active compound in a flavoured base, usually sucrose and acacia or tragacanth; and pastilles comprising the compound in an inert base such as gelatin and glycerin or sucrose and acacia.

Compositions of the present invention suitable for parenteral administration conveniently comprise sterile aqueous preparations of the conjugates of the invention, which preparations are preferably isotonic with the blood of the intended recipient. These preparations are preferably administered intravenously, although administration may also be effected by means of subcutaneous, intramuscular, or intradermal injection. Such preparations may conveniently be prepared by admixing the compound with water or a glycine buffer and rendering the resulting solution sterile and isotonic with the blood. Injectable formulations according to the invention generally contain from 0.1% to 60% w/v of active compound and are administered at a rate of 0.1 ml/minute/kg.

Formulations suitable for rectal administration are preferably presented as unit dose suppositories. These may be prepared by admixing the conjugates with one or more conventional solid carriers, for example, cocoa butter, and then shaping the resulting mixture.

Formulations or compositions suitable for topical administration to the skin preferably take the form of an ointment, cream, lotion, paste, gel, spray, aerosol, or oil. Carriers which may be used include Vaseline, lanoline, polyethylene glycols, alcohols, and combination of two or more thereof. The active compound is generally present at a concentration of from 0.1% to 5% w/w, more particularly from 0.5% to 2% w/w. Examples of such compositions include cosmetic skin creams.

Formulations suitable for transdermal administration may be presented as discrete patches adapted to remain in intimate contact with the epidermis of the recipient for a prolonged period of time. Such patches suitably contain the active compound as an optionally buffered aqueous solution of, for example, 0.1 M to 0.2 M concentration with respect to the said active compound. See for example Brown, L., et al. (1998).

Formulations suitable for transdermal administration may also be delivered by iontophoresis (see, for example, Panchagnula R, et al., 2000) and typically take the form of an optionally buffered aqueous solution of the active compound. Suitable formulations comprise citrate or Bis/Tris buffer (pH 6) or ethanol/water and contain from 0.1 M to 0.2 M active ingredient.

Formulations suitable for inhalation may be delivered as a spray composition in the form of a solution, suspension or emulsion. The inhalation spray composition may further comprise a pharmaceutically acceptable propellant such as carbon dioxide or nitrous oxide.

The conjugates may be provided in the form of food stuffs, such as being added to, admixed into, coated, combined or otherwise added to a food stuff. The term food stuff is used in its widest possible sense and includes liquid formulations such as drinks including dairy products and other foods, such as health bars, desserts, etc. Food formulations containing compounds of the invention can be readily prepared according to standard practices.

Therapeutic methods, uses and compositions may be for administration to humans or animals, including mammals such as companion and domestic animals (such as dogs and cats) and livestock animals (such as cattle, sheep, pigs and goats), birds (such as chickens, turkeys, ducks), fish and other marine organisms, and the like.

The conjugates or pharmaceutically acceptable derivatives, for example prodrugs or salts thereof, can also be co-administered with other active materials that do not impair the desired action, or with materials that supplement the desired action, such as antibiotics, antifungals, antiinflammatories, or antiviral compounds. The conjugates can comprise further drugs in combination or as a synergistic mixture.

The co-administration may be simultaneous or sequential. Simultaneous administration may be effected by the compounds being in the same unit dose, or in individual and discrete unit doses administered at the same or similar time. Sequential administration may be in any order as required and typically will require an ongoing physiological effect of the first or initial active agent to be current when the second or later active agent is administered, especially where a cumulative or synergistic effect is desired.

Without being limited to any one mode or principle, it is postulated that upregulation of a biotin receptor other than the sodium dependent multi-vitamin transporter (SMVT) might be responsible for the efficacy of the conjugates of the invention. It is generally accepted that uptake of biotin occurs through the SMVT, which permits co-transport of only small molecules, whose size is considerably less than that of the conjugates of the invention. This suggests that uptake of conjugate-bound biotin may be due to another different biotin binding surface protein/receptor, working in collaboration or independently from, the SMVT. The inventors have also found that the intracellular fate of biotin, once internalized, is different from either vitamin B12 or folate. As such, the intracellular processing of biotin-drug conjugates may be different from both Vitamin B12- or folate-targeted conjugates. This receptor profile and/or intracellular processing may thus contribute to one or more improved properties of the conjugates of the invention.

The present invention is further described with reference to the following examples which are in no way limiting on the scope of the invention.

### EXAMPLES

### Example 1

### Synthesis of Multi-Lysine Polymer 1 (MLP1)

A multi-lysine polymer (MLP1) of the formula [(NH₂-Gly)₄-Lys₂-Ser₂-Lys]₅-Ala-COOH, was synthesized on an Applied Biosystems peptide synthesiser. More precisely this represents [(NH₂-Gly)₄-Lys₂-Ser₂-Lys]₄[Gly₄-Lys₂-Ser₂-Lys]-Ala-COOH

The formula [(NH₂-Gly)₄-Lys₂-Ser₂-Lys]₄[Gly₄-Lys₂-Ser₂-Lys]-Ala-COOH can be represented as follows: 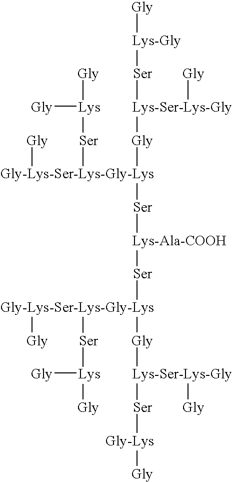
which show the structure more precisely.

### Example 2

### Synthesis of Multi-Lysine Polymer 2 (MLP2)

A multi-Lysine polymer (MLP2) of the general formula [(NH₂-Gly)₁₆-Lys₈-Lys₄-His₄-Glu₄-Lys₂-Lys]-Gly₅-Cys-COOH was synthesized on an Applied Biosystems peptide synthesiser. More precisely the structure can be represented as follows:

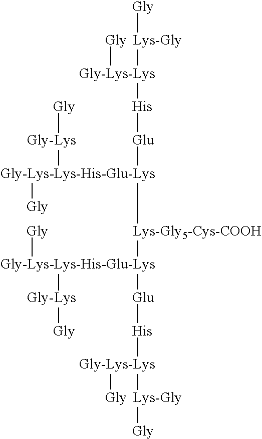

### Example 3

### Preparation of NHS-Biotin

Biotin (5 g) was dissolved in 100 ml dry dimethyl sulfoxide (DMSO), plus 2.5 ml triethylamine.

N-hydroxysuccinimide (2.6 gm) was added as a powder to the biotin and reacted overnight with 4.7 gm dicyclohexylcarbodiimide at room temperature. The dicyclohexylurea was removed by filtration. The DMSO was concentrated under reduced pressure and heating, and NHS-biotin precipitated with diethylether.

The product was washed several times with anhydrous ether, dried under vacuum and stored as a white powder.

### Example 4

### Formation of MLP-toxin conjugates using biodegradable cross-linkers.

There are many toxins which could be used for formation of biotin-MLP-toxin conjugates, including momordin, Pseudomonas exotoxin A, ricin and abrin. A general method for the formation of biotin-MLP-toxin conjugates is described below:

Conjugates are prepared in which the covalent linker contains a biodegradable disulfide bond, which would be reduced in vivo, presumably by intracellular glutathione in the tumor cell, thereby releasing the active substance after transport from the serum into the tumor cell. Briefly, MLP1 or MLP2 was reacted with N-succinimidyl 3-(2-pyridyldithio)propionate (SPDP). The dithiopyridyl-MLP (DTP-MLP) product was purified by RP-HPLC. A free thiol was introduced onto the toxin by a two step procedure in which the toxin was firstly reacted with SPDP, after which the thiopyridyl group was reduced with mercapto-ethanol. The product was purified by RP-HPLC. Alternatively free thiol was introduced into the toxin directly by reaction with iminothiolane. The thiolated product (SH-HN⁺toxin) was purified by RP-HPLC. Formation of the disulfide linked MLP-toxin conjugates was achieved by reaction of the thiolated toxin derivative with DTP-MLP in 2.5% acetic acid for 24 hours. The conjugated material was purified by Sephadex G-25 chromatography, followed by RP-HPLC.

### Example 5

### Preparation of Poly-Drug-HPMA-Biotin Conjugate.

Two N-(2-Hydroxypropyl)methacrylamide (HPMA) copolymers were synthesized as polymer backbones for the incorporation and derivatization with cytotoxic drugs and biotin. A non-biodegradable polymer backbone (HPMA-GG) was synthesized by the free radical copolymerization of HPMA with N-methacryloylglycylglycine p-nitrophenyl ester. A biodegradable polymer (HPMA-GFALG) was synthesized by the free radical copolymerization of HPMA with N-methacryloylglycylphenylalanylleucylglycine p-nitrophenol ester by the method of Rejmanova and co-workers [Rejmanova, P., Obereigner, B., and Kopecek, J. 1981 Makromol. Chem. 182:1899-1915]. In order to incorporate ricin A chain and biotin onto the polymers, they were reacted with a ten molar excess of a mixture of aminohexyl-biotin and Dithiopyridyldodecylsuberyl-hexylamine (1:10 mole:mole) overnight. Unreacted nitrophenyl esters were subjected to aminolysis by the addition of 1-amino-2-propanol. The modified polymers were purified by chromatography on Sepharose 6B. A solution of the dithiopyridyldodecylsuberylhexyl modified biotin-substituted polymers was dissolved in 2.5% acetic acid and reacted with ricin A chain. The reaction mixture was left for 144 hours at 4° C., afterwhich the ricin-biotin-substituted polymers were purified by chromatography on Sepharose 6B.

### Example 6

### Preparation of Poly-Daunomycin-HPMA-Biotin Conjugate

A N-(2-Hydroxypropyl)methacrylamide (HPMA) copolymer was synthesized as a polymer backbone for the incorporation and derivatization with both the cytotoxic drug, daunomycin and biotin. A biodegradable polymer (HPMA-GFLG) was synthesized by the free radical copolymerization of HPMA with N-methacryloylglycylphenylleucinylglycine p-nitrophenol ester by the method of Rejmanova and co-workers [Rejmanova, P., Obereigner, B., and Kopecek, J. 1981 Makromol. Chem. 182:1899-1915]. In order to incorporate daunomycin and biotin onto the polymers, they were reacted with a ten molar excess of a mixture of aminohexyl-biotin and daunomycin (1:10 mole:mole) overnight. Unreacted nitrophenyl esters were subjected to aminolysis by the addition of 1-amino-2-propanol. The modified polymers were purified by chromatography on Sepharose 6B.

### Example 7

### Preparation of ¹²⁵I Labelled Polymers

Bolton-Hunter reagent was dissolved at 1 mg/ml in DMSO. The amino-derivatized polymer was dissolved at 5 mg/ml in DMSO or DW containing 25 μl/ml DIEA. A 3 μl aliquot of Bolton-Hunter was added to 20 μl of the polymer solution. The reaction was allowed to proceed for 3 hours. Unreacted Bolton-Hunter was extracted with DCM (5×100 μl) after addition of 50 μl water. ¹²⁵I(1 μl) was added to the derivatized polymer, followed by the addition of 4 μl Chloramine-T dissolved at 20 mg/ml in PBS. The reaction proceeded for 15 secs, at which time the radioactive polymer was purified on PD10 column which had been equilibrated with 2.5% AcOH.

### Example 8

### Alternative Method of Preparation of Hydroxypropylmethacrylamide (HPMA)

1-Amino-2-propanol (58 g) was dissolved in acetonitrile (225 ml). The solution was cooled to −10° C. using an ethanol/dry ice bath. Methacryloyl chloride (40 g) in acetonitrile (170 ml) was added dropwise with vigorous stirring from a pressure equalising dropping funnel. The mixture was then allowed to warm slowly to room temperature overnight. The hydrochloride salt of 1-amino-2-propanol was removed by filtration through Celite filter aid. The solvent was removed at reduced pressure with a bath temperature of 50° C. The product was isolated by dissolving in methanol and precipitation using acetone. The product was then dissolved in DW and dialysed extensively against DW.

### Example 9

### Preparation of Amino-HPMA

HPMA (4.0 g) was dissolved in DMSO (100 ml). A 1.5 ml aliquot of DIEA was added followed by 1.26 gm of solid CDI (1,1′-carbonyldiimidazole). The HPMA was activated for 45 min, whereupon an excess of 1,6-diaminohexane (4.0 g) was added. The reaction proceeded for 2 h, at which time the product was dialysed to remove unreacted amines. The final product was lyophilized.

### Example 10

### Preparation of Lysyl-HPMA

HPMA polymer (100K<MW<300K, 2.8 g) was dissolved in DMF (40 mL). DIEA (560 μL) was added, followed by Disuccinimidyl carbonate (1512 mg) and the mixture stirred at room temperature under N₂ overnight. Lysine was dissolved at 100 mg/ml in 10% sodium carbonate. 1 gm lysine was added to the derivatized-HPMA and allowed to react overnight. The product was purified by dialysis to remove free DSC and lysine.

### Example 11

### Preparation of Methotrexate-HPMA Polymers Targeted with Biotin

HPMA polymer (100K<MW<300K, 2.8 g) was dissolved in DMF (40 mL). DIEA (560 μL) was added, followed by Disuccinimidyl carbonate (1512 mg) and the mixture stirred at room temperature under N₂ overnight. Methotrexate-Gly-Phe-Leu-Gly-Lysine (630 mg) was added and the mixture stirred for 30 min.

Biotin-Lys (MW 372, 80 mg dissolved in 1% NaHCO₃ solution) was added and the mixture was reacted overnight. The Polymer-product was precipitated by the addition of ethyl acetate and the pellet collected by centrifugation at 5000 rpm. The pellet was washed twice with acetone, and the resultant product was dissolved in DW and dialysed extensively against ammonium hydrogen carbonate solution. The product was lyophilysed.

### Example 12

### Preparation of Methotrexate-Dextrin Polymers Targeted with Biotin

Dextrin polymer (100K<MW<300K, 2.8 g) was dissolved in DMF (40 mL). DIEA (560 μL) was added, followed by Disuccinimidyl carbonate (1512 mg) and the mixture stirred at room temperature under N₂ overnight. Methotrexate-Gly-Phe-Leu-Gly-Lysine (630 mg) was added and the mixture stirred for 30 min.

Biotin-Lys (MW 372, 80 mg dissolved in 1% NaHCO₃ solution) was added and the mixture was reacted overnight. The Polymer-product was precipitated by the addition of ethyl acetate and the pellet collected by centrifugation at 5000 rpm. The pellet was washed twice with acetone, and the resultant product was dissolved in DW and dialysed extensively against ammonium hydrogen carbonate solution.

The product was lyophilysed.

### Example 13

### Preparation of Aminohexyl-carboxymethyl Cellulose (CMC)

CMC (low viscosity) was dissolved at 25 mg/ml in DW (2 gm/40 ml). NHS (150 mg dissolved @ 100 mg/ml in acetone) was added followed by 300 mg dry EDAC. The CMC was reacted for 15 minutes, whereupon 5 ml 1 M diaminohexane pH 9.5 was added and allowed to react O/WE. The product was dialysed exhaustively against DW. The product was then filter sterilized.

### Example 14

### Biotin Derivatisation of Polymers

Biotin (90 mg) was dissolved in DMSO (5.0 ml). DIEA (75 μL) was added, followed by TSTU ((O-(N-Succinimidyl)-N,N,N′,N′-bis(tetramethylene)uronium hexafluorophosphate) (180 mg). The biotin was activated for 10 min, then 1.0 g Polymer (amino-HPMA, or amino-hexyl-CMC) dissolved in DMSO (50 ml) was added to the activated biotin solution and reacted overnight. The product was dialysed extensively to ensure removal of unreacted acid. The product was lyophilized.

### Example 15

### Preparation of Methotrexate-GFLG-HPMA-Biotin

Methotrexate-GFLG-OH (FW 828, 36 mg, 3 ×biotin) was dissolved in DMSO (5 ml). DIEA (20 μL) was added, followed by TSTU (35 mg). The methotrexate was activated for 10 min. The polymer (100 mg) (Aminohexyl-HPMA or biotin-hexyl-HPMA) dissolved in DMSO (15 ml) was added to the activated Drug-GFLG-acid solution and reacted 60 min. The product was dialysed extensively to ensure removal of unreacted acid and lyophilysed.

### Example 16

### Preparation of Methotrexate-GFLG-CMC-Biotin

Methotrexate-GFLG-OH (FW 828, 36 mg, 3×biotin) was dissolved in DMSO (5 ml). DIEA (20 μL) was added, followed by TSTU (35 mg). The methotrexate was activated for 10 min. The polymer (100 mg) (Aminohexyl-CMC or biotin-hexyl-CMC) dissolved in DMSO (15 ml) was added to the activated Drug-GFLG-acid solution and reacted 60 min. The product was dialysed extensively to ensure removal of unreacted acid and lyophilysed.

### Example 15

### Preparation of Clorambucil-GFLG-HPMA-Biotin

Chlorambucil-GFLG-OH (FW 678, 29 mg, 3×biotin) was dissolved in DMSO (5 ml). DIEA (20 μL) was added, followed by TSTU (35 mg). The chlorambucil was activated for 10 min. The polymer (100 mg) (Aminohexyl-HPMA or biotin-hexyl-HPMA) dissolved in DMSO (15 ml) was added to the activated Drug-GFLG-acid solution and reacted 60 min. The product was dialysed extensively to ensure removal of unreacted acid and lyophilysed.

### Example 16

### Preparation of Chlorambucil-GFLG-CMC-Biotin

Chlorambucil-GFLG-OH (FW 678, 29 mg, 3×biotin) was dissolved in DMSO (5 ml). DIEA (20 μL) was added, followed by TSTU (35 mg). The chlorambucil was activated for 10 min. The polymer (100 mg) (Aminohexyl-CMC or biotin-hexyl-CMC) dissolved in DMSO (15 ml) was added to the activated Drug-GFLG-acid solution and reacted 60 min. The product was dialysed extensively to ensure removal of unreacted acid and lyophilysed.

### Example 17

### Preparation of HPMA-hexylaminosuccinate

Aminohexyl-HPMA (300 mg) was dissolved in DMSO (5 ml) and succinic anhydride (100 mg) and DIEA (100 μL) added. The polymer was reacted overnight then dialysed extensively against DW and lyophilysed.

### Example 18

### Preparation of Daunomycin-GLFG-HPMA-Biotin

HPMA-hexylaminosuccinic acid (35 mg) was dissolved in DMSO (2.0 ml). TSTU (18 mg) was added and activated for 10 min. H₂N-GFLG-Daunomycin (FW 938, 3×biotin, 4.4 mg) was added and allowed to react for 5 min. For targeted polymers 6-aminohexyl-biotin (3 mg, designed to give 20% loading) was added and reacted for 1 h. The product was dialysed to remove unconjugated reagents. The final product was concentrated using an AMICON positive pressure stirred cell with 10K membrane.

### Example 19

### Preparation of MTX-GFLG-MLP-Biotin

MTX-GFLG-OH (FW 828, 25 mg) was dissolved in DMSO (2 ml). TEA (5 μl) was added, followed by TSTU (15 mg, 1.2 equiv.). The reaction was allowed to proceed for 10 min, afterwhich 13 mg MLP Polymer dissolved in DMSO (0.5 ml) was added and reacted for 60 min. For preparation of targeted polymers biotin (8 mg) dissolved in DMSO (0.8 ml) was activated with TSTU (8.5 mg) for 10 min and then the activated targeting agent was added to MTX-GFLG-MLP mixture. The reaction proceeded for 60 min. 0.1 M Tris pH 7.5(5 ml) was added and stirred 1 h. The product was dialysed extensively and lyophilysed.

### Example 20

### Demonstration of Biotin-Mediated Targeting of Polymers

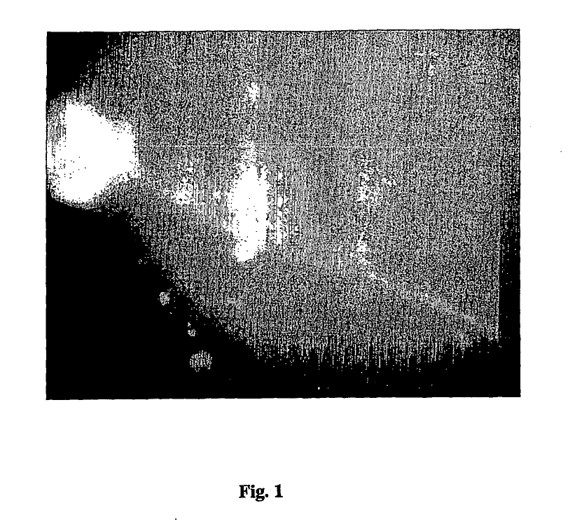

In order to examine the potential utility of biotin as a targeting agent for polymer-drug conjugates, Lysyl-HPMA was substituted with rhodamine using rhodamine-isothiocyanate using standard methods. An aliquot of the Rho-HPMA was then further reacted with biotin, to produce a biotin-substituted-Rhodamine-HPMA. Control polymers were prepared without biotin. For tumour accumulation studies, various strains of mice bearing a variety of tumours were injected intraperitoneally with 5 mg/kg Rhodamine conjugated to the HPMA polymers. Six hours after injection, the mice were sacrificed, their tumours removed and cryo-embedded before cryostatic sectioning. The level of accumulation of the Rhodamine-HPMA was determined by fluorescent microscopy using a Zeiss microscope equiped with Axioplan software. Representative sections are shown in FIG. 1. The data shows that the level of polymer uptake by P815 tumour cells can be enhanced by biotin derivatization of the rhodamine labelled polymers, as indicated by red staining. Blue staining is BisBenzamide staining of cell nuclei.

### Example 21

### Increased Localization of Targeted HPMA in L1210FR Tumour Cells in DBA/2 Mice with Biotin

Preliminary experiments were performed in L1210FR mice to determine whether Rhodamine-labelled polymers would localize to ascites cells in L1210 FR tumours injected IP.

Lysyl-HPMA was derivatized with Fluorescein (using FITC) or rhodamine (using TRITC) using standard methods. Derivatization was aimed at 5% substitution, however, with FITC this was too substituted and resulted in an insoluble polymer, therefore substitution was backed off to 2.5%. For the production of targeted polymers the Glycyl-5′O-VB₁₂, and folate were activated with TSTU and used to substitute the remaining amino groups on the fluorescent polymers. Polymers were also biotinylated with NHS-biotin. Free reagents were removed by dialysis.

Mice were injected IP with 100 ug polymer and left for 5 hours, at which time the mice euthanased by cervical dislocation. The peritioneal cavity was then flushed with 5 ml of 3.8% trisodium citrate, and ascites fluid, containing cells, was then aspirated from the peritioneal cavity. The fluid was kept at 4° C. ON before processing. The quantity of cells in the peritoneal wash out was determined by centrifuging the fluid and measuring the volume of the pellet. A fixed quantity of cells were then diluted out two-fold in an ELISA plate for measurement of fluorescence and determination of the level of uptake of fluorescent polymer.

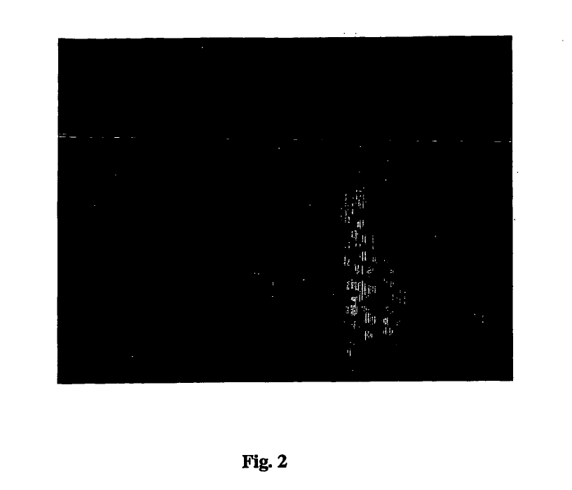

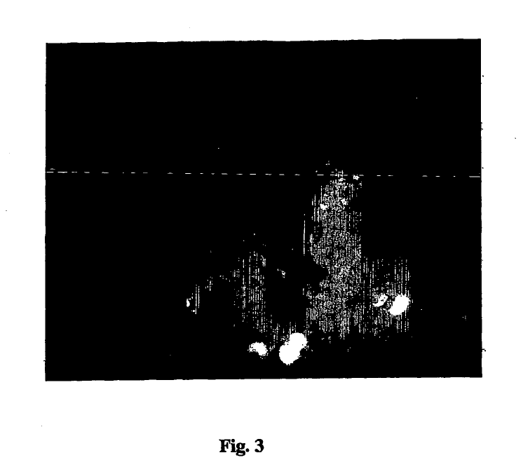

Cells were also placed on slides for microscopic examination of internalized fluorescence. Fluorescence determination was performed on a Zeiss Axioplan fluorescent microscope, and photographed. Results are shown in FIGS. 2. and 3. Examination of the amount of fluorescent polymer taken up by isolated ascites cells taken from mice at the time of sacrifice showed greatly increased uptake of all targeted polymers. Greatest uptake was seen with the biotinylated polymers, followed by folate and vitamin B12 as targeting agents.

### Example 22

### Preparation of Nanospheres

Nanospheres can be formed by a number of techniques common to those knowledgeable in the art, including: Solvent evaporation, Complex coacervation, Polymer/polymer incompatibility, Gelation, Interfacial polymerization and Thermal denaturation.

An effective amount of the complex is formulated with a pharmaceutically acceptable carrier, diluent or excipient to provide a medicament for administration to a patient requiring treatment of the conditions outlined in the body of the specification. The formulation is prepared using standard pharmaceutical techniques.

It is recognized that a number of factors will affect the determination of an appropriate dosage for a particular host. Such factors include the age, weight, sex, general health and concurrent disease states of the host. The determination of the appropriate dose level for the particular host is performed by standard pharmaceutical techniques.

### Example 23

### Preparation of Nanospheres by Coacervation

Almost any protein can be used as the matrix for entrapping drug via the desolvation technique, however preferred proteins according to the invention include bovine serum albumen (BSA), Ovalbumen (OA) and collagen.

BSA Nanospheres Formed by Desolvation.

Nanospheres were prepared by coacervation of BSA following desolvation, according to the method of Oppenheim (Oppenheim, 1984, Oppenheim et al 1984, 1982), Briefly a 40% ammonium sulphate solution was added dropwise to a solution of 1% BSA containing 0.5% Tween 20 and the turbidity monitored by Klett readings, until the turbidity rose rapidly. At this point (determined by experimentation) the solution was placed in an ultra-turrax and 600 ul of glutaraldehyde added to cross-link the nanoparticles. Cross-linking was stopped by the addition of a solution of 12% sodium metabisulfite. Particles were then washed extensively with distilled water prior to coupling to the NHS-derivative of biotin

### Example 24

### Incorporation of 5-fluorouracil

The antimitotic, 5-fluorouracil, was dissolved at 10 g/100 ml of the BSA/Tween solution. Desolvation and cross-linking was carried out as described in Example 23.

### Example 25

### Coupling of Biotin to Nanospheres

Proteinaceous nanospheres (prepared by the method described in Example 23) were surface coated with biotin by reaction of biotin with EDAC and NHS followed by addition to the preformed nanospheres.

### Example 26

### Preparation of Biotin-Lipid Complexes for Hydrophobic Insertion into Nanospheres

In order to link biotin to the surface of nanospheres which have no readily available chemical groups suitable for chemical conjugation, it is possible to prepare a complex of biotin to an hydrophobic moiety which can insert, non-covalently, into the surface of the nanospheres. Such a molecule is easily added at the time of formation of the nanospheres. The strength of the hydrophobic association is such that there is only a very slow dissociation of the biotin from the nanospheres under physiological conditions.

a) Preparation of biotin-phosphatidyl ethanolamine (biotin-PEA)

Phosphatidylethanolamine (100 mg) was dissolved in 2 ml chloroform/methanol (50:50, v/v). Biotin (100 mg) was added to the mixture. The biotin was then cross-linked to the PEA by the addition of 200 mg of the carbodiimide, 1-Ethyl-3-(3-Dimethylaminopropyl)carbodiimide (EDC or EDAC). The reaction was allowed to proceed for 90 minutes prior to the addition of the biotin-PEA to nanospheres.

b) Preparation of other complexes between biotin and an hydrophobic moiety.

Covalent complexes can be made between analogues of biotin and almost any aliphatic or aromatic chains or amphipathic containing a water soluble head group suitable for conjugation and a lipid soluble tail suitable for hydrophobic association within an hydrophobic environment. Thus, any lipid (saturated, unsaturated or polyunsaturated) which has a carboxylic acid head group, such as Oleic acid, octanoic acid, linoleic acid or glycerophophoric acids may be directly conjugated to an amino-biotin derivative using a suitable carbodiimide (EDAC or DCC, for example). Similarly any amphiphathic molecule possessing an amino-group (amino-hexane, amino-decane, amino-dodecane, phosphatidyl-ethanolamine, may be conjugated directly to carboxy-biotin using carbodiimides.

### Example 27

### Preparation of Biotin-Nanospheres by Solvent Evaporation

a) Preparation of biotin-PEA-[Polymethylmethacrylate] nanospheres

Polymethylmethacrylate (PMM, Polysciences)(MW 12,000; 500 mg) was dissolved in 2 ml of dichloromethane (DCM). The PMM in DCM was then added dropwise to 20 ml of 0.25% Polyvinylalcohol (PVA) while homogenizing at 13,500 rpm with a Janke & Kunkel Ultraturrax. After 1 minute, 200 ul of biotin-PEA was added and stirred gently overnight. The nanospheres were then harvested by centrifugation, washed three times with water and lyophilized.

b) Preparation of biotin-[PEA-Poly-lactic acid] nanospheres.

Poly-lactic acid (PLA, Polysciences)(MW 50,000; 500 mg) was dissolved in 3 ml of DCM and then homogenized into 20 1% PVA at 13,500 rpm on Ultraturrax T25 with an S25F probe for 5 minutes. biotin-PEA (400 ul) was added while the solution was stirred gently. Nanospheres were harvested as described above.

c) Preparation of biotin-PEA-[Poly-Hydroxy-butyrate/valerate] nanospheres

Poly-Hydroxy-butyrate/valerate (9% valerate) (ICI; 500 mg) was dissolved in 5 ml of DCM and homogenized into 20 ml 1% PVA at 13,500 rpm on Ultraturrax T25 with an S25F probe for 5 minutes. biotin-PEA (400 ul) was added and the spheres processed as described in 8b.

### Example 28

### Covalent Conjugation of Biotin to Nanospheres with Surface Carboxyl Groups

A general method for the conjugation of biotin to the surface of nanospheres made from polymers with free carboxyl groups is outlined below. The specific example utilizes commercially available carboxyl-modified nanospheres.

Polysciences Fluoresbrite™ carboxylate Nanospheres (2.5% Solids Latex) were obtained from Polysciences in sizes of 0.045 um, 0.49 um, 2.2 um and 9.97 um. One ml of each of the preparations was washed extensively with DW and resuspended in 200 ul of distilled water. To each preparation was added 1.5 mg aminohexyl biotin then 5 mg of EDAC. Each preparation was allowed to react overnight, after which unreacted material was removed by repeated washing with DW or by dialysis against DW.

### Example 29

### Surface Derivatization of Nanospheres

Many polymers used in the preparation of nanospheres by solvent evaporation do not contain functional groups for direct conjugation to biotin or its functionalized analogues, however it is possible to modify the surface of the preformed nanospheres to introduce functional groups suitable for conjugation to biotin.

a) Surface derivatization of Polylactic acid (PLA) nanospheres

Preformed PLA nanospheres (10 mg) were gently suspended in distilled water (DW; 350 ul) by rotation on a rotary shaker for 2 hours. Hydrazine hydrate (10 ul) was added and the suspension was shaken overnight at room temperature. The spheres were spun down and repeatedly washed with water by re-suspension and centrifugation. The washing procedure was repeated until the supernatant failed to give a positive hydrazine test (purple colour upon reaction with a solution of TNBS; 1 mg /ml). The spheres were washed a further two times and the wet pellet used directly for conjugation to biotin.

b) Conjugation of biotin to hydrazine modified PLA nanospheres

A sample of the hydrazine modified PLA nanospheres (3 ul wet pellet) was suspended in DW (250 ul). Aqueous solutions of biotin (10 mg/ml, 400 ul) and EDAC (100 mg/ml, 100 ul) were added and the reaction mixture shaken overnight at room temperature. The suspension was spun down and the supernatant removed. The pellet was washed repeatedly with DW (6 washes). The residual pellet, was vacuum dried.

Two control reactions were performed concurrently with the above conjugation. In the first a 3 mg sample of hydrazine-modified PLA nanospheres was treated with the biotin as described above but DW was used in place of the EDAC solution. In the second control a 2 mg sample of unmodified PLA nanospheres was treated with both biotin and EDAC as described above. For both controls the pellet remaining after repeated washing was a clear white colour with no evidence of any associated biotin.

### Example 30

### Preparation of Isobutyl-Cyanoacrylate Nanocapsules, Surface-Coated with Biotin

Nanocapsules suitable for biodistribution studies were prepared with ¹²⁵I-insulin as an internal marker. Briefly, 10 mg insulin was dissolved at 10 mg/ml in 0.1 M HCl. An aliquot (1 μl) of ¹²⁵I-insulin was added to the cold insulin, which was mixed with 100 μl Miglyol™ and vortexed. EtOH (10 ml) was added to the insulin/Miglyol™ mix and mixed by vortexing. IBCA (100 μl, Sicomet) was added to the clear solution, which was immediately added to 60 ml 0.25% F-127. After 30 minutes the preparation was split into 2 equal halves. One half was left to stir overnight, whilst to the other half was added 27 mg biotin-PEG-octadecanoic acid (80 mg/ml in EtOH). The solution was left to stir overnight. Both solutions were then treated in a similar fashion. Large aggregates were removed by centrifugation at 10K for 20 minutes. Both particle preparations were concentrated and washed in a Amicon positive pressure filtration unit using a 300,000 MW cut off membrane. Particles were stabilized by surface cross-linking with di-succinimidyl-2-aminoethyl-2-amino-2-benzyl-ethanoate (DSAB). DSAB was converted to the NHS-ester as follows. DSAB (40 mg) was dissolved in an equal weight of DMF, to which was added NHS (24 mg, 240 μl DMF). DCC (Dicyclohexylcarbodiimide, 44 mg, 440 μl, made up fresh) was then added to the DSAB mixture and allowed to activate for 20′ while stirring rapidly. The DSAB-NHS-ester was added at 0.32 mg per 2.1 mg nanocapsules, and left to stir O/N. The particles were then dialysed before use in biodistribution studies.

### Example 31

### Identification of Cells that Over-Express Receptors Involved in Vitamin Uptake

Cells from various tumour cell lines were allowed to grow for 2 days on glass slides incubated in appropriate media at 37° C. in 5% CO₂. After 2 days the media was removed and was replaced with spent culture media containing a Rhodamine-HPMA polymer to which was bound vitamin B12, folate or biotin. Cells were incubated for a further 5 hours at 37° C. in 5% CO₂. At this time the media was removed and uptake into the cells was assessed by fluorescent microscopy of internalization of the rhodamine fluorophore. Uptake was determined on a relative scale.

TABLE 1
Over-expression of vitamin receptors amongst various Tumour Cell lines

| Tumour | | | Polymer | Folate | VB12 | Biotin |
|---|---|---|---|---|---|---|
| O157 | Balb/C | Bcell Lymph | +/− | +/− | +/− | +/− |
| BW5147 | AKR/J | Lymphoma | +/− | +/− | +/− | +/− |
| B16 | C57/BI | Melanoma | − | − | − | − |
| LL-2 | C57/BI | Lung | − | − | − | − |
| HCT-116 | Balb/C-Nu | Colon Carcin | + | − | − | − |
| L1210 | DBA/2 | Leukemia | − | +/− | +/− | − |
| L1210FR | DBA/2 | Leukemia | − | ++ | + | +++ |
| Ov 2008 | Balb/C-Nu | Ovarian | − | +++ | − | ++ |
| ID8 | C57/BI | Ovarian | − | +++ | − | ++ |
| Ovcar | | Ovarian | − | +++ | − | ++ |
| Colo-26 | Balb/C | Colon Carcin | − | +/− | ++ | +++ |
| P815 | DBA/2 | Mastocytoma | − | +/− | ++ | +++ |
| M109 | Balb/C | Lung | − | + | +++ | +++ |
| RENCA | Balb/C | Renal cell | − | + | +++ | +++ |
| 4T1 | Balb/C | Breast | − | + | +++ | +++ |
| JC | Balb/C | Breast | − | + | +++ | +++ |
| MMT060562 | Balb/C | Breast | − | + | +++ | +++ |

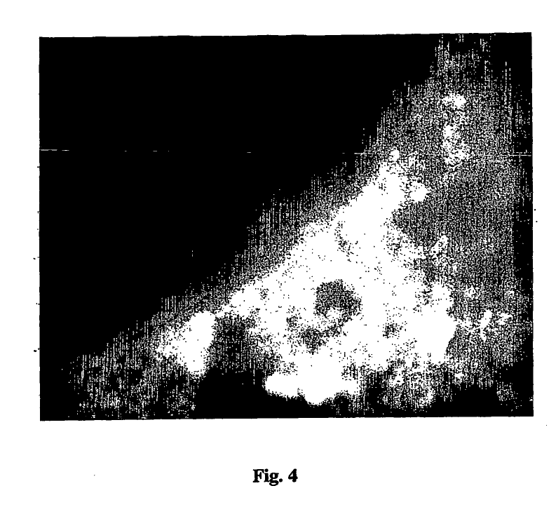

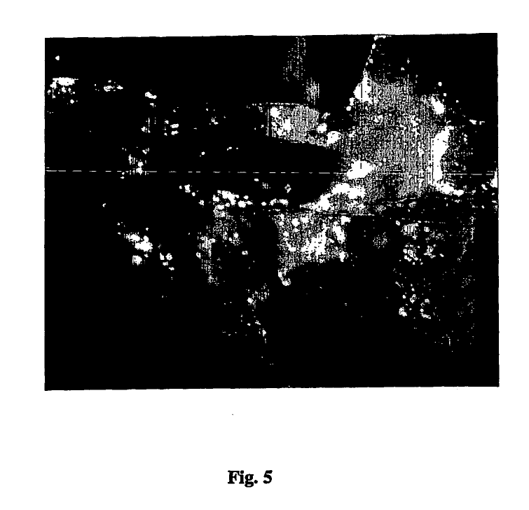

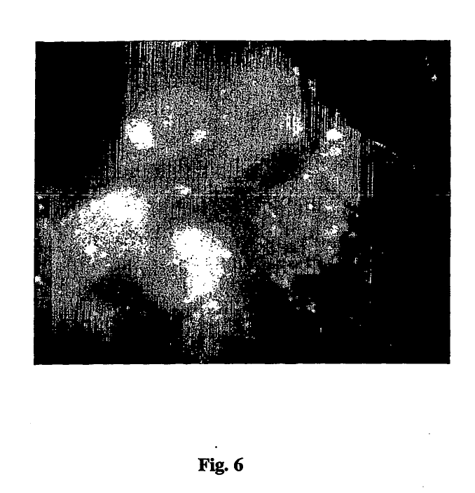

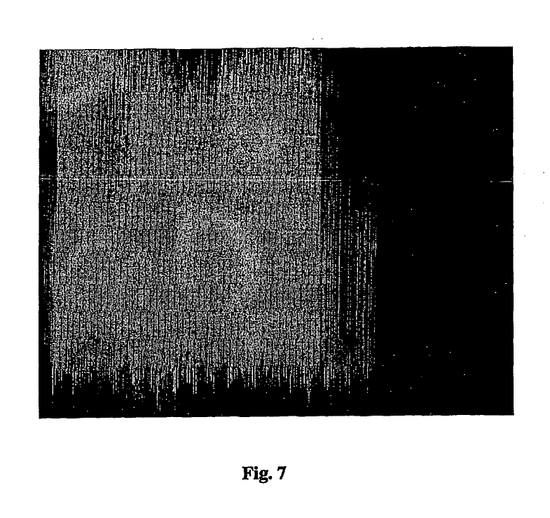

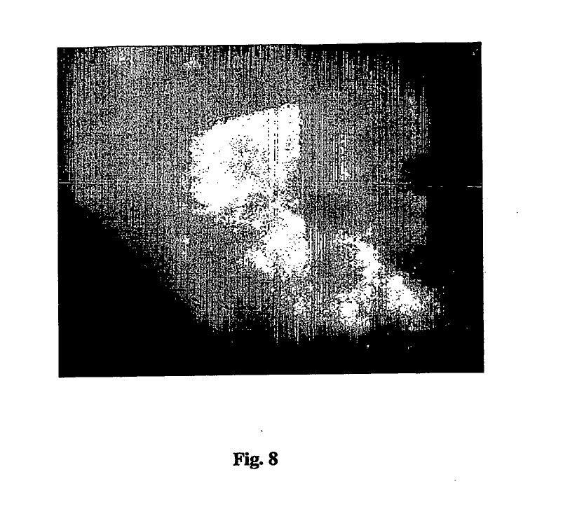

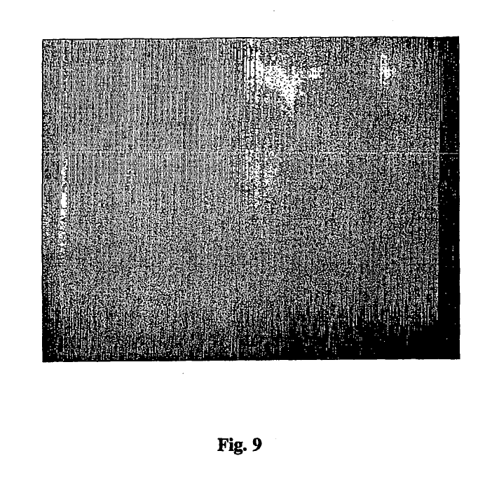

As can be seen from Table 1, all tumours that over-expressed receptors involved in Vitamin B12, or folate uptake, also over-expressed receptors involved in biotin binding. Representative FIGS. 4 to 9 show uptake into Ov2008, RENCA, 4T1, JC and MMTO60562 are also attached.

### Example 32

### Enhanced Killing of Colo-26 Tumour Cells Treated with Dox-GFLG-HPMA Targeted with Biotin

Dox was covalently linked to C-terminus of the tetrapeptide NH₂Gly-Phe-Leu-Gly-COOH as described above. The tetrapeptide-Dox conjugate was then linked to the HPMA polymer, after which the polymer was modified with the targeting agents biotin, folate and vitamin B₁₂. Non-conjugated material was removed by extensive dialysis. The Colo-26 tumour (2×10⁶ cells) was injected into Balb/C mice and allowed to grow for 7 days, at which time a small lump was apparent at the site of subcutaneous injection of the tumour. Mice were then injected intravenously with a dose of 20 mg/kg doxorubicin, either alone or conjugated to the polymer, on each of 3 successive days. The tumour was then allowed to grow in the mice, and its size determined via a two way measuremtn using Venier calipers. Data is presented as the average tumour weight of the mice over time.

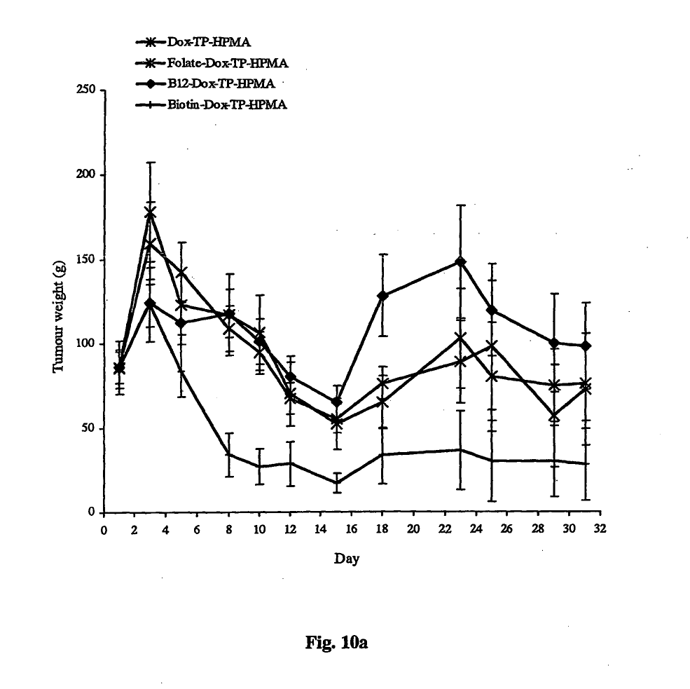

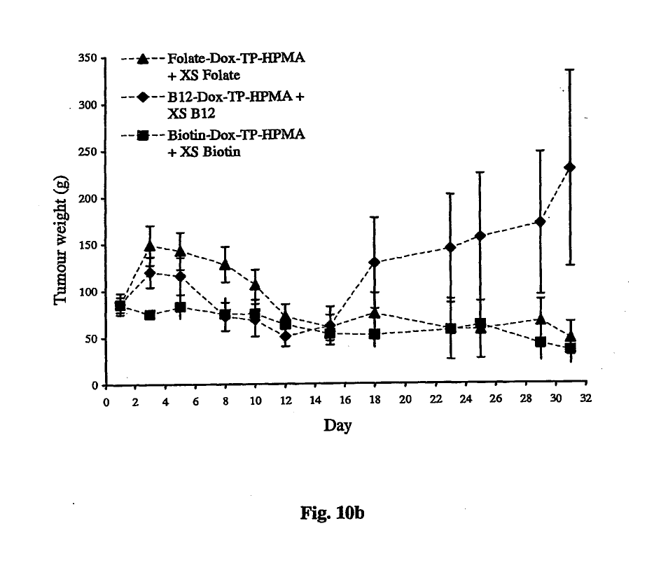

As can be seen from FIG. 10, substantial reduction in tumour mass was seen in the group that received the biotin-targeted Dox-TP-HPMA. The biotin dependency of this increased killing was shown by the reduced efficacy of this polymer conjugate in the presence of excess biotin. In fact the biotin-targeted polymer group was the only group that showed enhanced killing over and above that seen with the polymer alone.

### Example 33

### Preparation of DNM-HPMA Hydrazone

Oxidised-PHPMA (3000 mg) was dissolved in MeOH (30 mL). Hydrazidyl-DNM (300 mg) was added to each aliquot, followed by 1 Drop AcOH. The mixture was stirred for 3 h, after which, Hydrazidyl biotin (95 mg) was added and the mixture was then stirred overnight. The product then precipitated upon addition of ethyl acetate and was isolated by centrifugation. The pellet was washed with acetone and again isolated by centrifugation. The pellet was dissolved in PBS and dialysed at pH 7.4.

### Example 34

### Preparation of Biotin-poly(HPMA)-GGG-Ame

Poly(HPMA)-GGG-Ame (7.5 g) was dissolved in DMSO (75 mL) and TEA (400 μL) was added. DSC (disuccinimidyl carbonate, 405 mg) was then added and the mixture stirred for 24 h at room temperature (22° C.). AE-Biotin (MW 286, 260 mg) was added and the mixture stirred for a further 1 hour. Ethyl acetate (4 volumes) was added to precipitate the polymer and the mixture spun at 5000 rpm for 10 min and the supernatant removed. Acetonitrile (4 volumes) was added to resuspend the polymer, after which the mixture was spun at 5000 rpm for 10 min and the supernatant removed. The pellet was dissolved in distilled water and purified by tangential flow filtration, at which time the mixture was lyophilysed from water/MeCN.

### Example 35

### Preparation of Biotin-poly(HPMA)-GG-Ame

PHPMA-GG-ONp (KBT196-200A, 100 mg) was dissolved in DMSO (1 mL) and AE-Biotin (3.0 mg) added. The mixture was stirred for 1 h before addition of Hydrazine.2HCl (100 mg) dissolved in 3 mL MeOH containing TEA (0.5 mL). Targeted polymer was then added to the solution of hydrazine, which was reacted for 2 h. The resultant product was diluted with DW and dialysed extensively against ammonium hydrogen carbonate solution and then DW. Product was lyophilysed.

### Example 36

### Conjugation of Dox to Biotin-poly(HPMA)-GG-Ame Hydrazone

The biotin-targeted PHPMA bearing hydrazidyl functionality was dissolved in MeOH (1.0 mL) and DNM (10 mg) added plus 1 Drop AcOH. The mixture was stirred for 2 days and the product then precipitated upon addition of ethyl acetate. The pellet was washed with acetone and again isolated by centrifugation. The pellet was dissolved in PBS and dialysed at pH 7.4.

### Example 37

### Preparation of Dox-TP-Lysyl-poly(HPMA)

HPMA polymer (1.5 g) was dissolved in DMF (30 mL) and DIEA (250 μL) added. Disuccinimidyl carbonate (250 mg) was added and the mixture stirred at room temperature under N₂ overnight. Lys-Succ-GFLG-DNM (MW 1174, 200 mg) was then added and the mixture stirred for 30 min. Biotin-Lys (MW 372, 50 mg) was added to the solution which was then allowed to react overnight. The resultant product was diluted with DW and dialysed extensively against ammonium hydrogen carbonate solution. The product was lyophilysed.

### Example 38

### Preparation of HYNIC-HPMA Suitable for Use with ^{99m}Tc

HPMA-GFLG-en polymer (AT-119-134, 4.0 g) was dissolved in DMSO (20 mL) and Boc-HYNIC-OSu (Succinimidyl 6-BOC-hydrazinonicotinate, MW 350, 300 mg) added. The mixture was stirred for 1 h. Separately, biotin (400 mg) was dissolved in 6.0 ML DMSO, DIEA (240 μL) was added, followed by TSTU (520 mg) and the mixture activated for 15 min. The activated vitamin was added to the HPMA-GFLG-en-HYNIC-Boc prepared above, and stirred for 2 h. Free amino groups were blocked by the addition of a solution of acetic anhydride (60 μL) in DMSO (500 μL) containing NHS (70 mg). The mixture was stirred for 2 h. The product was precipitated by addition of ethyl acetate, and isolated by centrifugation at 5000 rpm for 10 min. The pellet was washed by sonication in MeCN and again isolated by centrifugation. This pellet was then dissolved in TFA (20.0 mL) and after 20 min the product precipitated on addition of petroleum ether/ethyl acetate (100 mL). The pellet was washed by sonication in MeCN/ethyl acetate/light petroleum and again isolated by centrifugation. The pellet was then washed with acetone and spun at 5000 rpm for 5 min. The resultant pellet was redissolved in carbonate buffer and the polymer was dialysed extensively using MWCO 3500. The product was then lyophilysed.

### Example 39

### Preparation of Mtx-poly(HPMA)

HPMA polymer (8.0 g) was dissolved in DMSO (120 mL), to which DIEA (2000 μL) and Disuccinimidyl carbonate (2000 mg) were added sequentially, and the mixture stirred at room temperature under N₂ overnight. MTX-GFLG-Lys (1600 mg, α, γ mixture) was added and the mixture stirred for 30 min. The reaction mixture was divided into 4 aliquots. AH-VB₁₂ (MW 1497, 360 mg), FA-Lys (MW 569, 137 mg) and Biotin-Lys (MW 372, 89 mg) were added to separate aliquots. Water was added to aid solubility. The mixtures were reacted overnight. The resultant product was dissolved in DW and dialysed extensively against ammonium hydrogen carbonate solution then DW. The product was lyophilysed.

### Example 40

### Preparation of Mtx-GFLG-Lys poly(HPMA)

MTX-(OMe)-GFLG-OH (FW 842, 180 mg) dissolved in DMSO (4 mL), to which was added DIEA (30 μL) followed by HPPyU (95 mg). The material was activated for 15 min prior to addition to 900 mg Polymer (Lys-HPMA) dissolved in DMSO (20 mL). The reaction proceeded for 60 min, at which it was divided into 4 aliquots in preparation for addition of targeting agents.

### Example 41

### Preparation of VB12/folate/biotin-[Mtx-GFLG-Lys poly(HPMA)]

Separate aliquots of VB₁₂-Gly acid (100 mg) or FA (30 mg) or Biotin (MW 244, 17 mg) were dissolved in DMSO (1.0 mL), to which was added DIEA (10 μL) and pyBOP (43 mg). The solution was activated for 25 min, before addition of the activated acids to the Mtx-GFLG-Lys poly(HPMA) polymer aliquot. The reaction proceeded for 2 h before the addition of 0.1 M NaOH solution to pH 11 to remove methyl ester. Deprotection proceeded for 20 min, at which time it was dialysed extensively and then lyophilysed.

### Example 42

### Preparation of Poly(HPMA)-GFLG-en-Biotin

Biotin (MW 244, 250 mg) was dissolved in DMSO (3 mL) and TEA (150 μL) was added prior to addition of TSTU (MW 301, 308 mg) and activation for 15 min. Separately, PHPMA-GFLG-en (22 kDa, AT-119-134, 1.0 g) was dissolved in DMSO (7 mL). The activated biotin was added to the rapidly stirring PHPMA-GFLG-en solution and the reaction was stirred for 4 h. The product was diluted with distilled water and dialysed extensively against DW (MWCO 3400). The dialysed solution was lyophilysed to afford the biotinylated polymer as a slightly brown powder.

### Example 43

### Preparation of Biotin-Targeted (poly(HPMA)-GGG-Ama-Pt-DACH

An aliquot of (poly(HPMA)-GGG-Ama-Pt-DACH, 200 mg) was dissolved in DMF/MeOH (1:1, 2 mL) and DIEA (10 μL) was added, before addition of pyBOP (MW 520, 12.5 mg), with stirring. AE-Desthiobiotin (5.7 mg) was added to the aliquot and the mixture stirred for 2 h before precipitation of the product by addition of ethyl acetate.

After centrifugation the pellet was sonicated in acetone and isolated again by centrifugation. The product was dissolved in DW and purified using Centricon 20's (5 kDa membrane) spinning at 4000 rpm for 30 min. The pellet was washed 3 more times. The product was lyophilysed.

### Example 44

### Preparation of poly(HPMA)-GFLG-en Succ-DNM

HPMA-GFLG-AE (HPMA-GFLG-en, AT-119-64, 600 mg) was dissolved in DMSO (5 mL). Succinyl-DNM (MW 627, 100 mg) was dissolved separately in DMSO (1 mL) and DIEA (20 μL) was added. HPPyU (70 mg) was added and the acid was activated for 15 min. The activated acid was added to HPMA-GFLG-en solution and reacted for 1 h. The mixture was divided into 3×2 mL aliquots for subsequent targeting.

### Example 45

### Preparation of VB12/FA-en-GLFG-HPMA-GFLG-en-Succ-DNM

Either VB₁₂-Gly-OH (MW 1456, 75 mg) or FA (MW 441, 22 mg) was dissolved in DMSO (500 μL) and DIEA (9 μL) was added. HPPyU (22 mg) was added and the acid was activated for 15 min. Activated acid was added to HPMA-GFLG-en-Succ-DNM solution and reacted for 2 h. The product was diluted with water and dialysed extensively

### Example 46

### Preparation of HPMA-GFLG-en-Mtx

HPMA-GFLG-AE (HPMA-GFLG-en, AT-119-64, 3000 mg) was dissolved in DMSO (40 mL). MTX (MW 454, 250 mg) was dissolved separately in DMSO (5 mL) and DIEA (200 μl) was added. pyBOP (340 mg) was added and the acid was activated for 55 min. Activated acid was added to HPMA-GFLG-en solution and reacted for 1 h. The mixture was divided into 4 aliquots for subsequent targeting.

### Example 47

### Preparation of VB_{12/}FA/Biotin-en-HPMA-GFLG-en-Mtx

Either VB₁₂-Gly-OH (MW 1456, 125 mg) or FA (MW 441, 40 mg) or Biotin (MW 244, 21 mg) was dissolved in DMSO (1500 μL) and DIEA (20 μL) was added. TSTU (31 mg) was added and the acid was activated for 15 min. Activated acid was added to an aliquot of HPMA-GFLG-en-MTX and reacted for 2 h. The product was precipitated with ethyl acetate and collected by centrifugation. The pellet was dissolved in water and dialysed extensively. Product was dialysed.

### Example 48

### Preparation of Mtx-HSA

Mtx was dissolved at 100 mg/ml in DMSO (88 mg). PyBOP (100 mg/ml in DMSO, 114 mg) plus 176 μl DIEA was added to the Mtx, and allowed to react for 60 minutes. HSA was dissolved at 100 mg/ml in 1% NaHCO₃ (880 mg), and the activated Mtx added to it and allowed to react overnight. The free Mtx was separated from Mtx-BSA on Sephacryl S-200 in PBS, before dialysis and lyophilization of the product.

### Example 48

### Preparation of Biotin-Modified Mtx-HSA

Biotin was dissolved at 100 mg/ml in DMSO. TSTU, dissolved at 130 mg/ml in DMSO, was added to the biotin as well as 100 μl TEA. The biotin was activated for 30 mins, before addition to Mtx-HSA (100 mg/ml in 1% sodium bicarbonate).

### Example 49

### Preparation of Dox-DSP-HSA

Doxorubicin (Dox) was dissolved at 100 mg/ml in DMF. A 4-molar excess of DSP was added to the Dox and allowed to react for 30 minutes. The product was precipitated with acetonitrile to 80%, resuspended in DMF and added at 5% w/w to HSA dissolved at 100 mg/ml in 1% NaHCO3. The material was allowed to react O/N, and was purified by dialysis. The product was biotinylated as described previously.

### INDUSTRIAL APPLICATIONS

The present invention provides a simple and novel technique for the specific targeting of pharmaceuticals to tumour cells using polymers. This technique has commercial applications in enhancing the efficacy of current tumour treatments as well as potential applications in treatment of inflammatory conditions.

The invention has been described herein, with reference to certain preferred embodiments, in order to enable the reader to practice the invention without undue experimentation. However, a person having ordinary skill in the art will readily recognise that many of the components and parameters may be varied or modified to a certain extent without departing from the scope of the invention. Furthermore, titles, headings, or the like are provided to enhance the reader's comprehension of this document, and should not be read as limiting the scope of the present invention.

The entire disclosures of all applications, patents and publications, cited herein, if any, are hereby incorporated by reference.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually of collectively, and any and all combinations of any two or more of said steps or features.

The reference to any prior art in this specification is not, and should not be taken as, an acknowledgment or any form of suggestion that that prior art forms part of the common general knowledge in the field of endeavour.

The following U.S. Patents, foreign patents and applications and other references are incorporated herein by reference.

U.S. Patents and Patent Applications

| | | |
|---|---|---|
| Low et al | 5,416,016 | 1995 |
| Russell-Jones et al | 5,428,023 | 1995 |
| Russell-Jones et al | 5,449,720 | 1995 |
| Russell-Jones et al | 5,548,064 | 1996 |
| Russell-Jones et al | 5,589,463 | 1996 |
| Russell-Jones et al | 5,807,832 | 1998 |
| Russell-Jones et al | 5,869,466 | 1999 |
| Russell-Jones et al | 6,150,341 | 2000 |
| Russell-Jones et al | 6,159,502 | 2000 |
| Russell-Jones et al | 6,262,253 | 2001 |
| Grissom, C. B, et al. | 6,315,978 | 2001 |

Foreign Patents and Applications

| | | |
|---|---|---|
| McEwan et al | PCT WO0066091 | 2000 |
| Grissom, C. B. et al | U.S. Pat Appl | 2002 |
| | 20020049154 | |

Selected References

Oppenheim R. C. (1984) in “Polymeric Microparticles” (Guiot, P and Couvreur, P. Eds.) CRC Press, Boca Raton.

Oppenheim R. C., Gipps, E. M. Forbes, J. F. and Whitehead R. H. (1984) in “Nanospheres and Drug Therapy” (Davis, S. S., Illum, L., McVie, J. G. and Tomlinson, E. Eds) Elsevier Science Publishers B.V.

Oppenheim, R. C., Stewart, N. F., Gordon, L. and Patel, H. M. (1982) Drug Devel. Indust. Pharm. 8:531-546.

Allen, R. H. and Majerus, P. W. (1972) J. Biol. Chem. 247:7702-7717.

Yamada, R.-H and H. P. C. Hogenkamp. (1972) J. Biol.Chem. 247:6266-6270.

1. A macromolecular conjugate comprising a support to which is coupled at least one targeting molecule in association with an active substance, wherein said targeting molecule is biotin or an analogue thereof possessing binding activity to a biotin receptor.

2. A conjugate of claim 1, wherein the support is a polymer.

3. A conjugate of claim 1, wherein the support is a nanoparticle.

4. A conjugate of claim 1 having the general formula:
(B−Q)ₙ−P−(Q′−A)ₘ
wherein B is biotin or a derivative thereof which is a carrier that binds to a biotin receptor
n, the molar substitution ratio of B in the conjugate, is a number from 1.0 to about 50;
P is a pharmaceutically acceptable linear, branched or dendritic polymer;
A is a pharmaceutically or diagnostic active substance;
m is a number greater than 1.0 to about 1000; and
Q and Q′ are independently a covalent bond, or a spacer compound linking biotin, P and A by covalent bonds.

5. The conjugate according to claim 4, wherein at least one of Q and Q′ is a spacer compound which contains a biodegradable portion.

6. The conjugate according to claim 5, wherein said biodegradable portion is selected from a disulfide bond, ester linkage, a γ-glutamyl-ε-lysine linkage and a diazo bond, and Gly-Phe-Leu-Gly.

7. The conjugate according to claim 4, wherein n is from 1.0 to about 1.5 and m is from 2 to about 200, more preferably from about 10 to 100.

8. A conjugate according to claim 4, wherein P is a biodegradable polymer.

9. A conjuguate according to claim 8, wherein said biodegradable polymer is selected from a biodegradable carbohydrate polymer or a polymer of amino acids.

10. A conjugate according to claim 4, wherein P is a non-biodegradable polymer.

11. A conjugate according to claim 10, wherein said non-biodegradable polymer comprises biodegradable side chains for covalent linkage to an active substance.

12. A conjugate according to claim 4, wherein said polymer is selected from poly[N-(2-hydroxypropyl)-methacrylamide], dextran or dextran derivatives, chondroitan sulfate, water soluble polyurethanes formed by covalent linkage of PEG with lysine, poly(glutamic acid), poly(hydroxypropyl glutamine), branched chain polypeptides, carboxymethyl cellulose, dendrimers and PEG-dendrimers.

13. A polymer according to claim 12, wherein said polymer is a branched chain polypeptide optionally modified to provide multiple functional groups for coupling of an active substance.

14. A conjugate according to claim 5, wherein said spacer compound Q or Q′ has from 1 to 50 atoms in its backbone.

15. A conjugate according to claim 4, wherein said spacer is a diradical spacer comprising optionally substituted alkylene C₁₋₅₀ moiety optionally contained within the chain, double bonds, triple bonds, aryl groups and/or hetero atoms.

16. A conjugate according to claim 15, wherein said spacer compound is derived from disuccinimidyl suberate (DSS), bis(sulfosuccinimidyl) suberate (BSS), ethylene glycolbis(succinimidylsuccinate) (EGS), ethylene glycolbis(sulfosuccinimidylsuccinate) (Sulfo-EGS), p-amino-phenylacetic acid, dithiobis(succinimidylpropionate) (DSP), 3,3′-dithiobis(sulfosuccinimidylpropionate) (DTSSP), disuccinimidyl tartarate (DST), disulfosuccinimidyl tartarate (Sulfo-DST), bis[2-(succinimidyloxycarbonyloxy)-ethylene]sulfone (BSOCOES), bis[2-(sulfosuccinimidooxycarbonyloxy)-ethylene]sulfone (Sulfo-BSOCOES), dimethyl adipimidate.2 HCl (DMA), dimethyl pimelimidate.2 HCl (DMP), or dimethyl suberimidate.2 HCl (DMS).

17. A conjugate according to claim 15, wherein said spacer compound is thiol cleavable.

18. A conjugate according to claim 17, wherein said thiol-cleavable spacer is derived from N-succinimidyl 3-(2-pyridyldithio)propionate (SPDP), iminothiolane, sulfosuccinimidyl 6-[3-(2-pyridyldithio) propionamido]hexanoate (Sulfo-LC-SPDP), succinimidyl 6-[3-(2-pyridyldithio) propionamido] hexanoate (LC-SPDP), sulfosuccinimidyl 6-[α-methyl-α-(2-pyridyldithio) toluamido] hexanoate (Sulfo-LC-SMPT), 1,4-di[3′-(2′-pyridyldithio)propionamido]butane (DPDPB), 4-succinimidyloxycarbonyl-α-methyl-α-(2-pyridyldithio)-toluene (SMPT) or dimethyl 3,3′dithiobispropionimidate.2 HCl (DTBP).

19. A conjugate according to claim 1, wherein said active substance is a biologically active toxin or a part thereof.

20. A conjugate according to claim 19, wherein said toxin is selected from ricin, abrin, diphtheria toxin, modecin, tetanus toxin, mycotoxins, mellitin, α-amanitin, pokeweed antiviral protein and ribosome-inhibiting proteins, from wheat, barley, corn, rye, gelonin and maytansinoid.

21. A conjugate according to claim 1, wherein said active substance is an alkylating agent selected from chlorambucil, cyclophosphamide, melphalan, cyclopropane; anthracycline antitumor antibiotics such as doxorubicin, daunomycin, adriamycin, mitomycin C, [2-(hydroxymethyl)anthraquinone]; antimetabolites such as methotrexate, dichloromethatrexate: cisplatin, carboplatin, and metallopeptides containing platimun, copper, vanadium, iron, cobalt, gold, cadmium, zinc and nickel, DON, thymidine, pentamethylmelamin, dianhydrogalactitol, 5-Methyl-THF, anguidine, maytansine, neocarzinostatin, chlorozotocin, AZQ, 2′deoxycoformycin, PALA, AD-32, m-AMSA and misonidazole.

22. A conjugate according to claim 1, wherein the active substance is an imaging agent.

23. A conjugate according to claim 22, wherein the imaging agent is Rhodamine, fluorescein, Texas red, Acridine Orange, Alexa Fluor (various), Allophycocyanin, 7-aminoactinomycin D, BOBO-1, BODIPY (various), Calcien, Calcium Crimson, Calcium green, Calcium Orange, 6-carboxyrhodamine 6G, Cascade blue, Cascade yellow, DAPI DiA, DiD, Dil, DiO, DiR, ELF 97, Eosin, ER Tracker Blue-White, EthD-1, Ethidium bromide, Fluo-3, Fluo-4, FM1-43, FM4-64, Fura-2, Fura Red, Hoechst 33258, Hoechst 33342, 7-hydroxy-4-methylcoumarin, Indo-1, JC-1, JC-9, JOE dye, Lissamine rhodamine B, Lucifer Yellow CH, LysoSensor Blue DND-167, LysoSensor Green, LysoSensor Yellow/Blu, Lysotracker Green FM, Magnesium Green, Marina Blue, Mitotracker Green FM, Mitotracker Orange CMTMRos, MitoTracker Red CMXRos, Monobromobimane, NBD amines, NeruoTrace 500/525 green, Nile red, Oregon Green, Pacific Blue. POP-1, Propidium iodide, Rhodamine 110, Rhodamine Red, R-Phycoerythrin, Resorfin, RH414, Rhod-2, Rhodamine Green, Rhodamine 123, ROX dye, Sodium Green, SYTO blue (various), SYTO green (Various), SYTO orange (various), SYTOX blue, SYTOX green, SYTOX orange, Tetramethylrhodamine B, TOT-1, TOT-3, X-rhod-1, YOYO-1 or YOYO-3.

24. A conjugate according to claim 1, wherein the active substance is a radionuclide.

25. A conjugate according to claim 22 wherein the imaging agent is a radionuclide.

26. A conjugate according to claim 22 wherein the imaging agent is linked to a polymer.

27. A conjugate according to claim 22 wherein the imaging agent is incorporated within and/or coated on a surface of a nanoparticle.

28. A conjugate according to claim 4 in which the pharmaceutically acceptable polymer has the sequence of [(NH₂-Gly)₄-Lys₂-Ser₂-Lys]ₙ-Ala-COOH, where n=1 to 85.

29. A conjugate according to claim 4 in which the pharmaceutically acceptable polymer has the sequence of [(NH₂-X₀)₄-Lys₂-Y₂-Lys]ₙ-Zₘ-COOH, where n=1 to 85; m=1 to 10; o=1 to 10; where X is any amino acid, where Y is any amino acid, and where Z is any amino acid.

30. A conjugate according to claim 4 in which the pharmaceutically acceptable polymer has the sequence of [(NH₂-Gly)₁₆-Lys₈-Lys₄-His₄-Glu₄-Lys₂-Lys]ₙ-Glyₘ-Cys-COOH, where n=1 to 85; where m=1 to 10.

31. A conjugate according to claim 4 in which the pharmaceutically acceptable polymer has the sequence of [(NH₂-X)₁₆-Lys₈-Lys₄-Y₄-Z₄-Lys₂-Lys]ₙ-AAₘ-Cys-COOH, where n=1 to 85; where m=1 to 10; where X, Y, Z and AA represent any amino acid independent of each other.

32. A conjugate according to claim 4 wherein P is poly[N-(2-hydroxypropyl)-methacrylamide].

33. A conjugate according to claim 1 or claim 4, wherein the biotin analogue selected from iminobiotin, Biocytin hydrazide, Biotin hydrazide, biocytin, 5-(Biotinamido)pentylamine, Sulfo-NHS(n-Hydroxysuccinimidyl)-Biotin, Sulfo-HNS-hexanyl-biotin (Sulfo-NHS-LD-Biotin), NHS-Biotin, Pentafluorophenyl-biotin, Pentafluorophenyl-polyethylenoxide-biotin, NHS-biotin Trifluoroacetamide, NHS-Iminobiotin trifluoroacetamide, Maleimido-polyethylenoxide biotin, Maleimido-polyethylenoxide iminobiotin, desthiobiotin, and chloracetyl-biotin.

34. A conjugate according to claim 2, wherein the biotin or biotin analogue is electrostatically or covalently linked to the polymer.

35. A conjugate according to claim 3, wherein the biotin or biotin analogue physically coats a surface of the nanoparticle.

36. A conjugate according to claim 35, wherein the biotin or biotin analogue physically coats the surface of the nanoparticle via electrostatic bonding, hydrogen bonding or hydrophobic bonding.

37. A conjugate according to claim 3, wherein the biotin or biotin analogue is attached to the nanoparticle by covalent bonding.

38. A conjugate according to claim 1, wherein the biotin analogue has cytotoxic or anti-inflammatory activity.

39. A process for synthesising a polymeric conjugate, comprising one or more of the following steps:
a) reacting an active substance with a polymer to form said conjugate;
b) chemically modifying the active substance to provide at least one functional group capable of forming a chemical linkage, and reacting the active substance and polymer to form said conjugate;
c) chemically modifying a target molecule, which is biotin or an analogue thereof, to provide at least one functional group capable of forming a chemical linkage and reacting the target molecule and polymer to form said conjugate;
d) chemically modifying the active substance and the polymer to provide functional groups capable of forming a chemical linkage, and reacting the active substance and polymer to form said conjugate;
e) reacting the active substance with at least one cross-linking agent and reacting the active substance of polymer to form said conjugate;
f) reacting the target molecule with at least one cross-linking agent and reacting the polymer and target molecule to form said conjugate;
g) reacting the active substance and polymer with at least one cross-linking agent and reacting the active substance and polymer to form said conjugate;
h) reacting the active substance directly with a polymeric support to form an intermediate containing one or more molecules of the active substance linked to the polymer, and subsequently coupling the polymer-active substance intermediate to one or more target molecules;
i) coupling one or more target molecules to a polymeric support and subsequently reacting the carrier-polymer intermediate with one or more molecules of the active substance to give a final conjugate containing one or more molecules of the active substance.

40. A conjugate according to claim 1, wherein biotin or an analogue thereof is a first targeting molecule, further comprising one or more second targeting molecules, the second targeting molecules perform a helper function for biotin-binding reactions necessary for uptake and/or transport of biotin in a cell.

41. A conjugate according to claim 1, wherein biotin or an analogue thereof is a first targeting molecule, further comprising one or more second targeting molecules, wherein the second targeting molecules assist in release of the active substance from the conjugate in a cell.

42. A conjugate according to claim 1, wherein biotin or an analogue thereof is a first targeting molecule, further comprising one or more second targeting molecules, wherein the second targeting molecules promote a biological activity of the active substance.

43. A process for the production of a conjugate having the general formula
(B−Q)ₙ−P−(Q′−A)ₘ
wherein B, Q, P, Q′, A, n and m are as defined in claim 4, said process selected from any one or more of the following steps:
a) reacting A with P to form an intermediate conjugate, and thereafter reacting the intermediate conjugate with biotin;
b) reacting biotin with P to form an intermediate conjugate and therafter reacting the intermediate complex with A;
c) the process of step a) or step b) wherein one or more of biotin, P or A are modified to provide at least one functional group capable of forming a chemical linkage prior to coupling with the other reactants; and
d) reacting one or two of biotin, P or A with Q and/or Q′ prior to coupling with the other reactants.

44. A process according to claim 43 wherein Q and/or Q′ comprises an optionally substituted alkylene C₁₋₅₀ moiety optionally within the chain, double bonds, triple bonds, aryl groups, and/or hetero atoms.

45. A process according to claim 43 wherein Q′ is a cleavable cross-linking agent containing a disulfide bond.

46. A process according to claim 45 wherein the cross-linking agents are selected from disuccinimidyl suberate (DSS), bis(sulfosuccinimidyl) suberate (BSS), ethylene glycolbis(succinimidylsuccinate) (EGS), ethylene glycolbis(sulfosuccinimidylsuccinate) (Sulfo-EGS), p-amino-phenylacetic acid, dithiobis(succinimidylpropionate) (DSP), 3,3′-dithiobis(sulfosuccinimidylpropionate) (DTSSP), disuccinimidyl tartarate (DST), disulfosuccinimidyl tartarate (Sulfo-DST), bis[2-(succinimidyloxycarbonyloxy)-ethylene]sulfone (BSOCOES), bis[2-(sulfosuccinimidooxycarbonyloxy)-ethylene]sulfone (Sulfo-BSOCOES), dimethyl adipimidate.2 HCl (DMA), dimethyl pimelimidate.2 HCl (DMP), dimethyl suberimidate.2 HCl (DMS).

47. A process according to claim 43 wherein said spacer is selected from disuccinimidyl suberate (DSS), bis(sulfosuccinimidyl) suberate (BSS), ethylene glycolbis(succinimidylsuccinate) (EGS), ethylene glycolbis(sulfosuccinimidylsuccinate) (Sulfo-EGS), p-amino-phenylacetic acid, dithiobis(succinimidylpropionate) (DSP), 3,3′-dithiobis(sulfosuccinimidylpropionate) (DTSSP), disuccinimidyl tartarate (DST), disulfosuccinimidyl tartarate (Sulfo-DST), bis[2-(succinimidyloxycarbonyloxy)-ethylene]sulfone (BSOCOES), bis[2-(sulfosuccinimidooxycarbonyloxy)-ethylene]sulfone (Sulfo-BSOCOES), dimethyl adipimidate.2 HCl (DMA), dimethyl pimelimidate.2 HCl (DMP), dimethyl suberimidate.2 HCl (DMS).

48. A process according to claim 43 wherein said spacer is selected from N-succinimidyl 3-(2-pyridyldithio)propionate (SPDP), iminothiolane, sulfosuccinimidyl 6-[3-(2-pyridyldithio) propionamido]hexanoate (Sulfo-LC-SPDP), succinimidyl 6-[3-(2-pyridyldithio) propionamido]hexanoate (LC-SPDP), sulfosuccinimidyl 6-[α-methyl-α-(2-pyridyldithio) toluamido]hexanoate (Sulfo-LC-SMPT), 1,4-di[3′-(2′-pyridyldithio)propionamido]butane (DPDPB), 4-succinimidyloxycarbonyl-α-methyl-α-(2-pyridyldithio)-toluene (SMPT), dimethyl 3,3′dithiobispropionimidate.2 HCl (DTBP).

49. A process according to claim 45 wherein the cross-linking agents are selected from N-succinimidyl 3-(2-pyridyldithio)propionate (SPDP), iminothiolane, sulfosuccinimidyl 6-[3-(2-pyridyldithio) propionamido]hexanoate (Sulfo-LC-SPDP), succinimidyl 6-[3-(2-pyridyldithio) propionamido]hexanoate (LC-SPDP), sulfosuccinimidyl 6-[α-methyl-α-(2-pyridyldithio) toluamido]hexanoate (Sulfo-LC-SMPT), 1,4-di[3′-(2′-pyridyldithio)propionamido]butane (DPDPB), 4-succinimidyloxycarbonyl-α-methyl-α-(2-pyridyldithio)-toluene (SMPT), dimethyl 3,3′dithiobispropionimidate.2 HCl (DTBP).

50. A conjugate prepared by a process of claim 43.

51. A conjugate of claim 3, wherein the nanoparticle is prepared by solvent evaporation, complex coacervation, polymer/polymer incompatibility, gelation, interfacial polymerisation or thermal denaturation.

52. A conjugate of claim 3, wherein the nanoparticle is biodegradable.

53. A process for the production of a conjugate of claim 52, which process comprises one or more of the following steps:
a) reacting nanospheres with a targeting molecule to form the conjugate;
b) chemically modifying a targeting molecule to provide at least one functional group capable of forming a chemical linkage and reacting nanospheres and the modified targeting molecules to form the conjugate;
c) reacting nanospheres with at least one cross-linking agent to prepare “activated” nanoparticles which are reacted with a targeting molecule to form the conjugate;
d) reacting a targeting molecule with at least one cross-linking agent and reacting the nanospheres with the reacted targeting molecule to form the conjugate;
e) reacting nanospheres and a targeting molecule with at least one cross-linking agent to the conjugate;
f) reacting nanospheres with at least one cross-linking agent, reacting a targeting molecule with at least one cross-linking agent and reacting the reacted nanospheres and the reacted targeting molecule to form the conjugate; or
g) reacting a targeting molecule with at least one cross-linking agent to prepare an analogue which is reacted with a hydrophobic moiety to form a hydrophobic derivative of the targeting molecule, and then incubating the hydrophobic derivative of the targeting molecule with a nanosphere in such a manner that the nanosphere is coated hydrophobically with the targeting molecule.

54. A process of claim 53, wherein the cross-linking agent contains a disulfide bond or is cleavable by acid, base or periodate.

55. A process of claim 53, wherein the cross-linking agent is selected from the group consisting of N-(4-azidophenylthio)phthalimide, 4,4′-dithiobisphenylazide, dithiobis(succinimidylpropionate), dimethyl-3,3′-dithiobispropionimidate.2HCl, 3,3′-dithiobis-(sulfosuccinimidylpropionate), ethyl-4-azidophenyl)-1,3′dithiopropionate, sulfosuccinimidyl-2-(m-azido-o-nitrobenzamido)-ethyl-1,3′-dithiobutyrimidate.HCl, N-succinimidyl-(4-azidophenyl)-1,3′dithiopropionate; sulfosuccinimidyl-2-(m-azido-o-nitrobenzamido)-ethyl-1,3′-dithiopropionate, sulfosuccinimidyl-2-(p-azidosalicylamido)-ethyl-1,3′-dithiopropionate, N-succinimidyl-3-(2-pyridylthio)propionate, sulfosuccinimidyl-(4-azidophenyldithio)-propionate, 2-iminothiolane, disuccinimidyl tartrate and bis-[2-(succinimidyloxycarbonyloxy)ethyl]-sulfone.

56. A process of claim 53, wherein the targeting molecule is cross-linked to the nanosphere or nanoparticle by reaction of the carrier with a carbodiimide and N-hydroxysuccinimide (NHS), and then reacting the NHS derivative with a suitable functional group on the nanosphere.

57. A process of claim 53, wherein the cross-linking agent contains a biodegradable bond.

58. A process of claim 57, wherein the cross-linking agent is cleaved by an esterase, glutathione, or azo-reductase.

59. A conjugate prepared by a process of claim 53.

60. A method for the modification of a polymeric support to introduce functional groups capable of reacting either directly with an active substance or with a chemically-modified form of the active substance, wherein a resulting polymer-active substance intermediate contains one or more molecules of the active substance, said intermediate being suitable for coupling to biotin or an analogue thereof to give a conjugate capable of amplified delivery of the active substance.

61. A pharmaceutical composition which comprises a conjugate according to any one of claims 1 to 21, 24 or 28-38 together with a pharmaceutically acceptable carrier or excipient.

62. A diagnostic imaging composition comprising a conjugate according to any one of claims 22 to 27.

63. A method for the treatment or prophylaxis of disease which comprises administering to a subject a therapeutically effective amount of a conjugate according to any one of claims 1 to 21, 24 or claim 28-38 or a composition of claim 61.

64. A method of claim 63 wherein the disease is cancer.

65. A method of claim 63, wherein the disease is an inflammatory condition.

66. A method of claim 65, wherein the disease is rheumatoid arthritis.

67. A method of claim 65, wherein the disease is Crohn's disease.

68. A method of claim 65, wherein the disease is inflammatory bowel disease.

69. A method of claim 63, wherein the disease is multiple sclerosis.

70. Use of a conjugate according to any one of claims 1 to 21, 24, or 24 to 28 in the manufacture of a medicament.

71. Use of a conjugate according to any one of claims 22 to 27 in the manufacture of a diagnostic imaging agent.

72. A method for the diagnosis of a pathological condition which comprises administering to a subject an effective amount of a conjugate according to claim 22 to 27 or a composition according to claim 62.

73. A conjugate according to claim 4, wherein the linker is biodegradable.

74. A conjugate according to claim 4, wherein the linker is a hydrazone.

75. A conjugate according to claim 4, wherein the linker contains 5-benzoyl-valeric acid.

76. A conjugate according to claim 4, wherein the linker is biodegradable and contains a valine-citrilline dipeptide.

77. A conjugate according to claim 4, wherein the linker is biodegradable and contains a phenylalanine-lysine dipeptide.

78. A conjugate of claim 1, wherein the active substance is a drug selected from platinum derivatives.

79. A conjugate according to claim 78, wherein the platinum derivative is selected from cis-Platin, CarboPlatin, oxaliplatin, multinuclear platinate species including BBR3464 and BBR3005, transdiamminedichloroplatinum (II) (Transplatin), chlorodiethylenetriammineplatinum (II), Platinum IV compounds, spiroplatin, platin-phosphine derivatives.

80. A conjugate of claim 1, wherein the active substance is doxorubicin or an analogue thereof, including daunorubicin, daunomycin, epirubicin, adriamycin.

81. A conjugate of claim 1, wherein the active agent is a cytotoxin selected from anti-folates including methotrexate and dichloromethatrexate.

82. A conjugate of claim 1, wherein the biotin is a hydrazidyl derivative of biotin.

83. A conjugate of claim 1, wherein the biotin is chloracetyl biotin.

84. A conjugate of claim 1, wherein the biotin is desthiobiotin.

85. A conjugate of claim 79, wherein the biotin is desthiobiotin.

86. A conjugate of claim 1, wherein the active substance is a dolastatin derivative.

87. A conjugate of claim 86, wherein the dolastatin derivative is auristatin or monomethylauristatin.

88. A conjugate of claim 4, wherein the linker is a valine-citrilline-aminobenzyl-carbamate derivative.
